(19) United States
(12) Patent Application Publication
(10) Pub. No.: US 2022/0406416 A1
(43) Pub. Date: Dec. 22, 2022

(54) Novel and efficient Graph neural network (GNN) for accurate chemical property prediction

(71) Applicant: University of Southern California, Los Angeles, CA (US)

(72) Inventors: Daniel Sylvinson Muthiah Ravinson, Los Angeles, CA (US); Mark E. Thompson, Anaheim, CA (US)

(21) Appl. No.: 17/843,341

(22) Filed: Jun. 17, 2022

Related U.S. Application Data
(60) Provisional application No. 63/212,301, filed on Jun. 18, 2021.

(51) Int. Cl. G16C 20/30; G16C 20/60; G16C 10/00; G16C 20/70

(52) U.S. Cl. CPC G16C 20/30; G16C 20/60; G16C 10/00; G16C 20/70

(57) ABSTRACT

A method for selecting a material having a desired molecular property comprises generating a combinatorial library of molecule structures derived from a core molecular structure, splitting the library into a training set configured to train a graph neural network (GNN) machine learning (ML) model, a test set configured to test the validity of and assess accuracy of the GNN model, and a prediction set where predictions are made using the GNN model, optimizing geometries of the molecular structures, computing excited state energies of the optimized geometries, encoding molecular structure information into a matrix, determining three mutually orthogonal principal axes, transforming spatial coordinates into mutually orthogonal coordinates, constructing a molecular graph with n nodes, feeding the molecular graph into the GNN model as an input, and selecting a material having a suitable desired molecular property based on the output of the GNN model.

### CROSS-REFERENCE TO RELATED APPLICATIONS

This application claims priority to U.S. provisional application No. 63/212,301 filed on Jun. 18, 2021, incorporated herein by reference in its entirety.

### BACKGROUND OF THE INVENTION

Machine learning (ML) has emerged as a useful tool aiding the advancement of virtually every field of science and technology. The current decade has seen an explosion of reports on the application of ML-based approaches in various aspects of materials design ranging from synthetic design to physical property prediction.¹⁻²⁵ The availability of large, structured databases and repositories that have consistently logged inorganic material properties and structures over several years have led to numerous studies employing ML approaches for inorganic solid-state materials design.²⁶⁻³¹ Studies exploring the application of ML methods in organic molecular materials design on the other hand have remained comparatively scarce but are growing rapidly.^{17, 18, 21, 32-38} A key aspect of developing data-driven ML solutions to the design process involves the utilization of ML algorithms to learn structure-property relationships from available data. Several challenges plague the development of a successful property-prediction ML workflow for organic materials like the lack of large, structured databases consistently cataloging structure-property relationships, morphological flexibility ranging amorphous/disordered to crystalline, inconsistency of electronic structure methods to name a few. These challenges are exacerbated for organic optoelectronic applications wherein viability of candidates is dependent on satisfying multiple narrowly defined criteria, therefore requiring extremely accurate property predictions. The parameters that are most critical for optoelectronic applications like OPVs, OLEDs etc. are energetic molecular properties like HOMO, LUMO and excited state (Sₙ, Tₙ) energies. Developing accurate ML models to predict these properties across large chemical libraries would significantly accelerate the discovery of promising candidates. In lieu of a widely accessible large-scale database listing experimentally derived optoelectronic properties of molecular materials, the alternative is to rely on electronic structure methods. Recently, databases containing DFT-predicted properties of compounds relevant for optoelectronics applications through projects like the Harvard Clean Energy Project³⁹ and the PubChemQC project⁴⁰ have been developed. The development of the QM7, QM8 and QM9 libraries containing a chemical universe of molecules with up to 7, 8 and 9 non-H atoms (C, O, N, F) respectively albeit less relevant for optoelectronic applications have served as a test bed for benchmarking various ML strategies.⁴¹⁻⁴⁵ A recent report demonstrated that chemical accuracy (^{˜}0.04 eV) could be reached for atomization energy predictions on the QM9 database using 0.7% of the database for training indicating that highly data-efficient models can indeed be developed for well-defined chemical subspaces.⁴⁶ Montavon et al. trained deep neural networks using coulomb matrices as descriptors to directly predict molecular properties based on QM data albeit on a small scale (7211 small molecules) and reported out of sample root mean square errors (RMSE) greater than 0.2 eV and 1.7 eV for MO energies and excitation energies respectively while using a training set that contained 70% of the database.⁴⁷ Ghosh et al. explored several deep neural net architectures including multilayer perceptron (MLP), convolutional neural network (CNN), and deep tensor neural network (DTNN) on a dataset of 132,000 molecules and noted that the prediction errors were still ^{˜}0.2 eV for MO energies despite using a training set that contains 90% of the dataset.⁴⁸ The SchNet deep learning architecture was able to achieve chemical accuracy for HOMO/LUMO energies using 84% of the QM9 database for training.¹⁴ Recently, Kang et al. reported random forest models using a combination of descriptors like extended connectivity fingerprints (ECFP), molecular access system (MACCS) keys, etc. to predict excitation energies of a subset of molecules in the PubChemQC database and the reported RMSE was still >0.4 eV, inadequate for any virtual screening strategy.⁴⁹ Alternatively, Ramakrishnan et al. proposed a hybrid approach referred to as the Δ-ML approach, wherein instead of directly predicting the absolute values of the molecular properties from the chemical descriptors, ML models are trained to recover the error differential between a low-level QM method like DFT and a more sophisticated method like CC2. Using this approach, the authors reported that excitation energies can be predicted at the level of accuracy of the CC2 method using TDDFT and the ML model trained on CC2 data from a fraction of the database.⁵⁰ The obvious downside of this approach is that these low-level calculations would still need to be performed on the whole database which becomes untenable for large-scale databases.

Given the apparent infinite size of the chemical universe, an exhaustive search of this space to identify compounds for one or more target applications appears seemingly impossible. Furthermore, the studies mentioned above demonstrate the challenge in developing a generalized ML model capable of predicting properties of entities in the entire chemical universe with sufficient accuracy due to the relative sparsity and/or lack of sufficient chemical diversity of any finite sub-library that may be developed to train such models. A further complication is that while ab initio methods like coupled-cluster, quantum Monte Carlo methods etc. can achieve predictive chemical accuracy (<0.04 eV), they become prohibitively expensive for medium-large systems relevant for most optoelectronic applications. Density functional theory (DFT) based methods can often serve as a compromise between accuracy and cost. Unfortunately, a fundamental problem with using DFT based methods to predict properties of diverse chemical spaces is the inexistence of a single universal DFT functional that can accurately predict molecular properties of all compounds in the chemical universe. This is even more of an issue for excited state properties, for example, TDDFT using a common hybrid functional like B3LYP can reliably predict excited state energies for most organic chromophores featuring simple localized transitions (π→π*, n→π*) but fails in systems featuring strong charge transfer (CT) transitions which require the use of range-separated hybrid (RSH) functionals with range separation (ω) parameters that may have to be tuned for each system based on the extent of CT.⁵¹⁻⁵⁶ Furthermore, there are certain classes of compounds like cyanine based chromophores which are of great import for optoelectronic applications, but whose excited state properties cannot be accurately captured by traditional TDDFT methods irrespective of the choice of functionals (errors>0.4 eV) on account of strong correlation effects and require more sophisticated treatments.⁵⁷

Thus there is a need in the art for improved novel and efficient graph neural networks (GNN) for accurate chemical property prediction.

### SUMMARY OF THE INVENTION

Some embodiments of the invention disclosed herein are set forth below, and any combination of these embodiments (or portions thereof) may be made to define another embodiment.

In one aspect, a method for selecting a material having a desired molecular property for optoelectronic applications comprising generating a combinatorial library of molecule structures derived from a core molecular structure based on a palette of chemical functionalities comprising at least one of a synthetic ease of access to all or most compounds in the generated library, an availability or synthesizability of precursors bearing the most possible combinations of the functionalities, and a chemical disparity or diversity of the functionalities within the palette, splitting the library into a training set configured to train a graph neural network (GNN) machine learning (ML) model, a test set configured to test the validity of and assess accuracy of the GNN model, and a prediction set where predictions are made using the GNN model, optimizing geometries of the molecular structures in the training set and test set via a semi-empirical, a molecular mechanics, a density functional theory (DFT), or an ab initio method, computing ground state and excited state properties via a semi-empirical, a molecular mechanics, a density functional theory (DFT), or an ab initio method, encoding molecular structure information associated with each molecular structure in the library into a matrix

$$M = [ \begin{matrix}{Z}_{1} {x}_{1} & {Z}_{1} {y}_{1} & {Z}_{1} {z}_{1} \\ ⋮ & ⋮ & ⋮ \\ {Z}_{n} {x}_{n} & {Z}_{n} {y}_{n} & {Z}_{n} {z}_{n}\end{matrix} ]$$

representing the chemical structure in an arbitrary cartesian coordinate system where Zᵢ, xᵢ, yᵢ, zᵢ represent the atomic number, x, y and z atomic spatial coordinates respectively, determining three mutually orthogonal principal axes (u, v, w) of the molecule by performing principal component analysis (PCA) on M, transforming the (x, y, z) spatial coordinates into the (u, v, w) mutually orthogonal coordinates via

$$R = [ \begin{matrix}{x}_{1}^{′} & {y}_{1}^{′} & {z}_{1}^{′} \\ ⋮ & ⋮ & ⋮ \\ {x}_{n}^{′} & {y}_{n}^{′} & {z}_{n}^{′}\end{matrix} ] = [ \begin{matrix}{x}_{1} & {y}_{1} & {z}_{1} \\ ⋮ & ⋮ & ⋮ \\ {x}_{n} & {y}_{n} & {z}_{n}\end{matrix} ] [ \begin{matrix}{u}_{1} & {v}_{1} & {w}_{1} \\ {u}_{2} & {v}_{2} & {w}_{2} \\ {u}_{3} & {v}_{3} & {w}_{3}\end{matrix} ] ,$$

constructing a molecular graph with n nodes each representing a constituent atom via encoding the (x′ᵢ, y′ᵢ, z′ᵢ) atomic coordinates as node features of the graph wherein the node features include an atomic identifier that encodes the kind of atom that the node represents, feeding the molecular graph into the GNN model as an input, providing the prediction set of molecule structures to the trained GNN model, and selecting a material having a suitable desired molecular property for optoelectronic applications based on the output of the GNN model.

In one embodiment, the method further comprises optimizing further the geometries of the molecular structures in the training set and test set via a density functional theory (DFT) method utilizing hybrid functional B3LYP with a 6-31G(d,p) basis set.

In one embodiment, the method further comprises optimizing further the geometries of the molecular structures in the training set and test set via a quantum chemistry method comprising a low-cost density functional theory (DFT), a Møller-Plesset perturbation theory (MP2), or a coupled cluster method.

In one embodiment, the method further comprises computing excited state energies of the optimized geometries of the molecular structures via an excited state quantum chemistry method comprising a time-dependent DFT (TDDFT), a Tamm-Dancoff approximation (TDA), an excited state coupled cluster approach, or a ΔSCF approach.

In one embodiment, the method further comprises computing S₁ energies via a restricted open-shell Kohn Sham (ROKS) ΔSCF approach.

In one embodiment, the method further comprises performing a grid search across a hyperparameter size to find the optimal model, wherein the hyperparameter comprises a number of GNN layers, a number of MLP layers, a number of nodes, an aggregation function, a batch size, and a learning rate.

In one embodiment, the method further comprises training via a stepwise approach the GNN model by taking the geometric encodings and the DFT computed properties of the molecules in the training set as inputs to learn the relationship between them.

In one embodiment, the method further comprises computing at each step the error metrics (MAE, R²) of the trained GNN model to perform predictions on the test set until a desired accuracy is reached or until the error metrics cease to improve appreciably.

In one embodiment, the core molecular structure comprises at least one of boron difluoride aza dipyridylmethene (DIPYR), boron difluoride aza diquinolylmethene (α-azaDIPYR), and Pentacene.

In one embodiment, the palette of chemical functionalities further comprises at least one of a highest occupied molecular orbital (HOMO), a lowest unoccupied molecular orbital (LUMO), an S₁ energy, and a T₁ energy.

In one embodiment, structural information associated with each molecule in the library is encoded into a feature vector to serve as an input to the GNN model, and wherein the feature vector includes at least one of an atom connectivity, a bonding pattern, and a 3D geometry.

In one embodiment, an effective featurization is learned on the fly during training.

In one embodiment, the (u,v,w) mutually orthogonal coordinates represent 3 mutually perpendicular molecular axes in the order of decreasing chemical variance from u through w.

In one embodiment, the atomic identifier includes the atomic number or a one-hot encoding vector of atom type.

In one embodiment, the node features scales linearly with system size.

In one embodiment, the molecular graph retains rotational, translational and permutational invariance.

In one embodiment, the number of GNN layers is from 1 to 20, the number of MLP layers is from 1 to 20, the number of nodes is from 1 to 2000, the aggregation functions include sums and averages, the batch size is from 1 to 100, and the learning rate is from 1 to 10⁻⁴.

In one embodiment, the size of the training set is from 1 to 500 molecules.

In another aspect, a method for selecting a material having a desired molecular property comprises generating a combinatorial library of molecule structures derived from a core molecular structure, splitting the library into a training set configured to train a graph neural network (GNN) machine learning (ML) model, a test set configured to test the validity of and assess accuracy of the GNN model, and a prediction set where predictions are made using the GNN model, optimizing geometries of the molecular structures in the training set and test set, computing excited state energies of the optimized geometries of the molecular structures, encoding molecular structure information associated with each molecular structure in the library into a matrix

$$M = [ \begin{matrix}{Z}_{1} {x}_{1} & {Z}_{1} {y}_{1} & {Z}_{1} {z}_{1} \\ ⋮ & ⋮ & ⋮ \\ {Z}_{n} {x}_{n} & {Z}_{n} {y}_{n} & {Z}_{n} {z}_{n}\end{matrix} ]$$

representing the chemical structure in an arbitrary cartesian coordinate system where Zᵢ, xᵢ, yᵢ, zᵢ represent the atomic number, x, y and z atomic spatial coordinates respectively, determining three mutually orthogonal principal axes (u,v,w) of the molecule by performing principal component analysis (PCA) on M, transforming the (x, y, z) spatial coordinates into the (u, v, w) mutually orthogonal coordinates via

$$R = [ \begin{matrix}{x}_{1}^{′} & {y}_{1}^{′} & {z}_{1}^{′} \\ ⋮ & ⋮ & ⋮ \\ {x}_{n}^{′} & {y}_{n}^{′} & {z}_{n}^{′}\end{matrix} ] = [ \begin{matrix}{x}_{1} & {y}_{1} & {z}_{1} \\ ⋮ & ⋮ & ⋮ \\ {x}_{n} & {y}_{n} & {z}_{n}\end{matrix} ] [ \begin{matrix}{u}_{1} & {v}_{1} & {w}_{1} \\ {u}_{2} & {v}_{2} & {w}_{2} \\ {u}_{3} & {v}_{3} & {w}_{3}\end{matrix} ] ,$$

constructing a molecular graph with n nodes each representing a constituent atom via encoding the (x′ᵢ, y′ᵢ, z′ᵢ) atomic coordinates as node features of the graph wherein the node features include an atomic identifier that encodes the kind of atom that the node represents, feeding the molecular graph into the GNN model as an input, providing the prediction set of molecule structures to the trained GNN model, and selecting a material having a suitable desired molecular property based on the output of the GNN model.

In another aspect, a system for selecting a material having a desired molecular property for optoelectronic applications comprises at least one database including data for a plurality of core molecular structures, and a computing system communicatively connected to the at least one database, comprising a processor and a non-transitory computer-readable medium with instructions stored thereon, which when executed by a processor, perform steps comprising generating a combinatorial library of molecule structures derived from a core molecular structure based on a palette of chemical functionalities comprising at least one of a synthetic ease of access to all or most compounds in the generated library, an availability or synthesizability of precursors bearing the most possible combinations of the functionalities, and a chemical disparity or diversity of the functionalities within the palette, splitting the library into a training set configured to train a graph neural network (GNN) machine learning (ML) model, a test set configured to test the validity of and assess accuracy of the GNN model, and a prediction set where predictions are made using the GNN model, optimizing geometries of the molecular structures in the training set and test set via a semi-empirical, a molecular mechanics, a density functional theory (DFT), or an ab initio method, computing ground state and excited state properties via a semi-empirical, a molecular mechanics, a density functional theory (DFT), or an ab initio method, encoding molecular structure information associated with each molecular structure in the library into a matrix

$$M = [ \begin{matrix}{Z}_{1} {x}_{1} & {Z}_{1} {y}_{1} & {Z}_{1} {z}_{1} \\ ⋮ & ⋮ & ⋮ \\ {Z}_{n} {x}_{n} & {Z}_{n} {y}_{n} & {Z}_{n} {z}_{n}\end{matrix} ]$$

representing the chemical structure in an arbitrary cartesian coordinate system where Zᵢ, xᵢ, yᵢ, zᵢ represent the atomic number, x, y and z atomic spatial coordinates respectively, determining three mutually orthogonal principal axes (u, v, w) of the molecule by performing principal component analysis (PCA) on M, transforming the (x, y, z) spatial coordinates into the (u,v,w) mutually orthogonal coordinates via

$$R = [ \begin{matrix}{x}_{1}^{′} & {y}_{1}^{′} & {z}_{1}^{′} \\ ⋮ & ⋮ & ⋮ \\ {x}_{n}^{′} & {y}_{n}^{′} & {z}_{n}^{′}\end{matrix} ] = [ \begin{matrix}{x}_{1} & {y}_{1} & {z}_{1} \\ ⋮ & ⋮ & ⋮ \\ {x}_{n} & {y}_{n} & {z}_{n}\end{matrix} ] [ \begin{matrix}{u}_{1} & {v}_{1} & {w}_{1} \\ {u}_{2} & {v}_{2} & {w}_{2} \\ {u}_{3} & {v}_{3} & {w}_{3}\end{matrix} ] ,$$

constructing a molecular graph with n nodes each representing a constituent atom via encoding the (x′ᵢ, y′ᵢ, z′ᵢ) atomic coordinates as node features of the graph wherein the node features include an atomic identifier that encodes the kind of atom that the node represents, feeding the molecular graph into the GNN model as an input, providing the prediction set of molecule structures to the trained GNN model, and selecting a material having a suitable desired molecular property for optoelectronic applications based on the output of the GNN model.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 depicts core molecular structures along with the palette of substitutions for exemplary libraries A, B and C in accordance with some embodiments.

FIG. 2 depicts exemplary GNN architecture in accordance with some embodiments.

FIG. 3 is a table depicting an exemplary comparison of DFT predicted values with experimentally reported values of relevant properties for related compounds in accordance with some embodiments.

FIG. 4 depicts an exemplary Schematic of a ML workflow used in accordance with some embodiments.

FIGS. 5A through 5C depict exemplary performance of different ML models with varying training set sizes for the 3 libraries in accordance with some embodiments.

FIG. 6 is a table depicting exemplary Error metrics of GNN models trained on 1000, 1500 and 2000 molecules for the 3 libraries on a test set of 450 structures each in accordance with some embodiments. The last 3 columns refer to the percentage of molecules in the test set featuring errors below 0.10, 0.15 and 0.20 eV.

FIGS. 7A through 7F depict exemplary simulation results in accordance with some embodiments. FIG. 7A shows a schematic of hybrid WOLED architecture explored. FIGS. 7B and 7C depict scatter plots of T1 and S1 energies predicted by the GNN(2000) models for libraries A and B respectively with the region of interest highlighted. FIG. 7D depicts a scatter plot of ML and DFT predicted HOMO and LUMO energies for selected candidates from A and B that satisfy the hybrid WOLED design criteria (based on ML predictions). FIGS. 7E and 7F depict distribution of ML and DFT predicted S1 and T1 energies of selected candidates from A and B respectively. Hollow circles indicate candidates with T2<S1 according to DFT calculations.

FIGS. 8A and 8B depict more exemplary simulation results in accordance with some embodiments. FIG. 8A depicts scatter density plots of S1 and T1 energies with the enclosing gray area indicating the SF parametric space wherein 0<S1−2T1<0.2 eV. FIG. 8B depicts DFT and ML predicted S1 and T1 energies of selected candidates with gray region as in FIG. 8A highlighting the space satisfying the SF criteria.

FIG. 9 depicts an exemplary computing environment in which aspects of the invention may be practiced in accordance with some embodiments.

### DETAILED DESCRIPTION OF THE INVENTION

It is to be understood that the figures and descriptions of the present invention have been simplified to illustrate elements that are relevant for a clearer comprehension of the present invention, while eliminating, for the purpose of clarity, many other elements found in systems and methods of novel and efficient graph neural networks (GNN) for accurate chemical property prediction. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

In recognition of the challenge presented above, disclosed herein is a more conservative yet practical approach wherein localized chemical subspaces combinatorially built by well-defined chemical modifications on one or few core chemical structures are explored. ML models are trained on a small yet sufficiently representative subset of this pre-defined subspace based on DFT/other QM methods that are known to predict relevant properties accurately for the class of molecules in this space. The core structures chosen to be explored would be ones that are, from a synthetic standpoint, amenable to a wide variety of chemical functionalization patterns across a large number of sites/positions to increase the likelihood of discovering candidates that satisfy design criteria for target applications. Analysis of pertinent literature reports and chemical intuition may also help guide the process of choosing core structures and the palette of chemical functionalities that define the exploration space. The practicality of this approach is augmented by the fact that the entire library may be accessible through one or few generalizable synthetic strategies. Demonstrated herein are libraries spanning millions of structures and a wide-spanning parametric design space can be generated using this approach. Furthermore predictive and actionable ML models are shown that can be developed for the generated libraries with minimal computational overhead at the accuracy requisite for practical screening strategies vis-à-vis optoelectronic applications.

Referring now in detail to the drawings, in which like reference numerals indicate like parts or elements throughout the several views, in various embodiments, presented herein are systems and methods for novel and efficient graph neural networks (GNN) for accurate chemical property prediction.

The systems, processes and methods described herein may be utilized for desired applications as would be appreciated by those skilled in the art. For example, practical applications include identifying material having a desired molecular property for optoelectronic applications, photovoltaics, lasers, bioimaging, electrochemical applications, redox chemistry, catalysis, or any other suitable application where ground state or excited state molecular properties are crucial.

The invention is described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out exemplary embodiments of the present invention, and are not to be construed as limiting in any way the remainder of the disclosure.

### Library Design

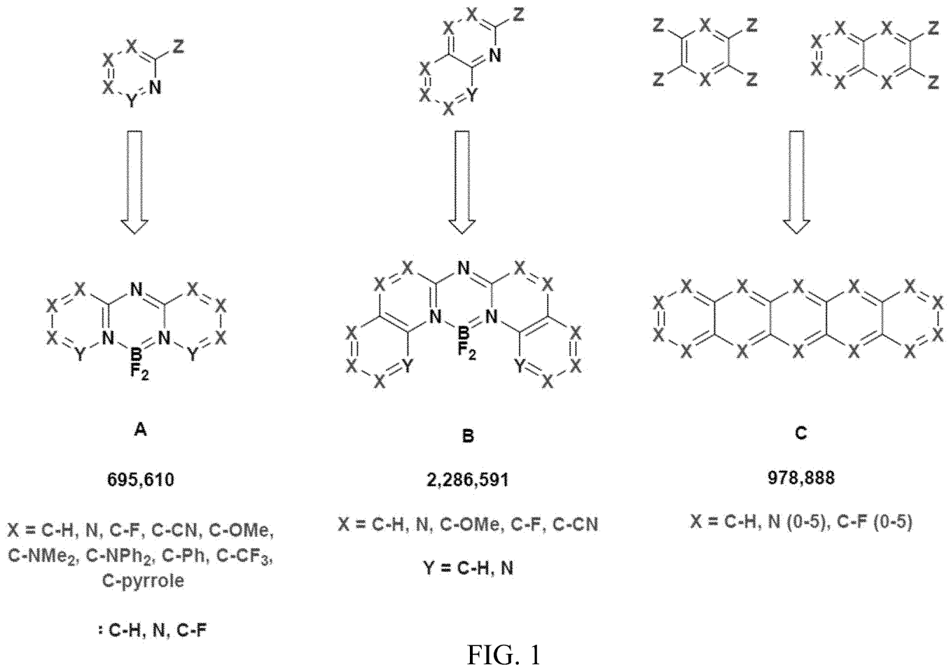

The libraries used in one embodiment were derived from three core structures: boron difluoride aza dipyridylmethene (DIPYR), boron difluoride aza diquinolylmethene (α-azaDIPYR) and Pentacene as depicted in the scheme of FIG. 1, and the corresponding libraries generated thereof will henceforth be referred to as A, B and C respectively. The azaDIPYR and α-azaDIPYR cores represent a relatively underexplored class of dyes that are pyridine and quinoline based analogues of the more popular boron dipyrromethene (BODIPY) based dyes that are widely used in numerous applications ranging photovoltaics^{58-60,} lasers⁶¹, bio-imaging⁶²⁻⁶⁵, etc. Reported dyes based on this core structure generally feature high extinction coefficients, high PLQY, sharp absorption and emission profiles like several of the BODIPY dyes lending themselves to optoelectronic applications like OPVs, OLEDs, etc.⁶⁶ However, despite their favorable properties, there have been very few reports of their employment in optoelectronics applications. The molecular properties of dyes based on these cores have been shown to be easily tunable by simple chemical modifications. For instance, the unsubstituted DIPYR compound exhibits green emission while substitution of a N atom or a CN functionality at the meso position shifts the emission to the blue region.^{67,68} Furthermore, it is possible to envision feasible synthetic routes (like Buchwald Hartwig couplings) to a large library of compounds built from a wide array of substitution patterns on the cores from readily available precursors. The palette of functionalities used to build combinatorial libraries were chosen based on 2 main considerations, the first being, synthetic ease of access to all/most compounds in the resulting library. This includes availability/synthesizability of precursors bearing most possible combinations of the functionalities. The second consideration is chemical disparity/diversity of functionalities within the palette. A more diverse palette would yield a more diverse chemical space, consequently widening the ambit of property space (ex. HOMO, LUMO, S₁, T₁, etc.) that may be accessed. Library A was built per the definition in the scheme of FIG. 1 with 9 different substituents covering a wide range of electro/nucleophilicity. The positions marked Y were restricted to only CH, aza- and fluoro-substitutions to avoid steric clashes with the BF₂ group of the core structure. The substituents on each pyridine ring are restricted to a maximum of 3 types to maintain synthetic feasibility. This yields a total of 695,610 unique structures. Library B based off the α-azaDIPYR core was built using a smaller palette of substituents: aza, methoxy, fluoro and cyano groups but across a higher number of sites. The Y sites were restricted to aza-substitutions to avoid steric clashes with the BF₂ group. A maximum of 2 different types of non-aza-substitutions and no more than 3 types including aza-substitutions are allowed for each quinoline ring. Further, the number of aza-substitutions on each of the α-rings is restricted to 2. These filters eliminate synthetically infeasible compounds. Upon application of these filters, a library containing 2,286,591 unique compounds is obtained.

Library C based on the pentacene core structure was built primarily with OPV applications in mind. Pentacene based structures are attractive due to their ability to undergo singlet fission and also the presence of a large n cloud make them potential non-fullerene acceptor candidates.^{69,70} The library was built using a palette of aza and fluoro-substitutions across 14 sites as depicted in the scheme of FIG. 1. These substitutions are known to stabilize the LUMO, making them more likely to serve as acceptor candidates for OPVs.⁷¹ The number of aza and fluoro substitutions are restricted to under 5 each, resulting in a total of 978,888 compounds.

Thus, the level of chemical diversity followed the order: A>B>C and library size follows the order B>C>A.

### QM Methods

The structures in the training and test sets of all 3 libraries were initially optimized using the PM7 semi-empirical as implemented in the MOPAC2016 package.⁷² The PM7 optimized geometries were then optimized using Density Functional Theory (DFT) using the B3LYP functional and 6-31G(d,p) basis set. All DFT calculations in one embodiment were performed using the Q-Chem 5.1 package.⁷³ Excited state energies were computed on the ground state optimized structures using Time-dependent DFT (TDDFT). The triplet excited states of the pentacene based structures were computed using the Tamm-Dancoff approximation (TDA)⁷⁴ while in all other cases full linear response TDDFT was used. Additionally, the S₁ energies of the cyanine-based structures (A and B libraries) were computed using the restricted open-shell Kohn Sham (ROKS) ΔSCF approach^{75,76} as implemented in Q-Chem.

### ML Models and Features

To develop ML models, the structural information associated with each molecule in the library like atom connectivity, bonding patterns, 3D geometry, etc. would need to be uniquely encoded into a feature vector to serve as inputs to an ML model. The ML task boils down to learning an accurate functional mapping from the feature vectors that encode chemical structure to the associated molecular properties. Several different types of molecular feature representations like extended connectivity fingerprints (ECFPs)⁷⁷, Coulomb matrices⁴⁴, bag of bonds⁷⁸ and connectivity counts⁷⁹ have been developed to train ML models for molecular property predictions. In this invention, the 12^{NP}3^{B} featurization that was recently reported by Collins et al. was used and was found to perform well for several molecular property prediction tasks especially for energetic parameters.⁷⁹ The features were generated for the molecules in the library using the molml 0.9.0 python library that was developed by the authors who proposed this approach. The features encode the 3D geometrical information as well as the bonding (single, double, etc.) and connectivity information of the molecules. More details regarding the approach and its implementation can be found in the paper published by Collins et al.⁷⁹

Two types of commonly used supervised ML methods mated with the 12^{NP}3^{B} featurization have been explored in one embodiment: Kernel ridge regression (KRR) and Gaussian processes (GP). KRR models based on Gaussian and Laplacian kernels were explored while GP models using the rational quadratic(RQ) and Matern kernels were developed for each library. These models were chosen in one embodiment on account of their ease of implementation and prior literature reporting their efficacy for similar molecular property prediction tasks.^{46, 50, 79} The mathematical forms of all 4 kernels are given below:

$$Gaussian kernel : k ( {x}_{i} , {x}_{j} ) = exp ( - {γ d ( {x}_{i} , {x}_{j} )}^{2} ) \\ Laplacian kernel : k ( {x}_{i} , {x}_{j} ) = exp ( - γ {‖ {x}_{i} , {x}_{j} ‖}_{1} ) \\ Rational Quadratic kernel ( RQ ) : k ( {x}_{i} , {x}_{j} ) = {( 1 + \frac{{d ( {x}_{i} , {x}_{j} )}^{2}}{2 {β l}^{2}} )}^{- β} Matern kernel : k ( {x}_{i} , {x}_{j} ) = \frac{1}{Γ ( v ) {2}^{v - 1}} {( \frac{\sqrt{2 v}}{l} d ( {x}_{i} , {x}_{j} ) )}^{v} {K}_{v} ( \frac{\sqrt{2 v}}{l} d ( {x}_{i} , {x}_{j} ) )$$

where β, α, γ, l and v are hyperparameters that will be tuned during training to find the optimal model in each case as described below. Kᵥ and Γ(v) are the modified Bessel function and gamma function, respectively. d(xᵢ,xⱼ) and ∥xᵢ,xⱼ∥₁ are the Euclidean and Manhattan distances.

The hyperparameters of the KRR models (γ and the regularization parameter, α) were tuned using a 5-fold cross-validation scheme using the training set across a 2D grid of values [10⁻¹⁵, 10⁻¹⁴, . . . , 10², 10³] for α and γ. The β and l hyperparameters for the RQ models were tuned across the range of values between 10⁻¹² and 10¹². Similarly, for the Matern models, the length scale hyperparameter (l) was tuned across a range between 10⁻¹² and 10¹² while two discrete values for v (0.5 and 1.5) were explored. All ML models reported here were implemented using the scikit-learn python library.⁸⁰

Graph neural networks (GNN) like the ones used in PhysNet¹³, SchNet^{14,81} etc. is another approach wherein an effective featurization is learnt on the fly during training and can therefore offer better performance. Most GNNs that encode 3D structural information use distance matrices or other approaches that scale exponentially with system size.⁸² Here, a simple linear scaling approach was implemented to encode 3D molecular structure uniquely and efficiently within a GNN framework. In order to uniquely represent the 3D structure of a molecule, a matrix M is first built that represents the chemical structure in some arbitrary cartesian coordinate system:

$$M = [ \begin{matrix}{Z}_{1} {x}_{1} & {Z}_{1} {y}_{1} & {Z}_{1} {z}_{1} \\ ⋮ & ⋮ & ⋮ \\ {Z}_{n} {x}_{n} & {Z}_{n} {y}_{n} & {Z}_{n} {z}_{n}\end{matrix} ]$$

where, Zᵢ, xᵢ, yᵢ, zᵢ represent the atomic number, x, y and z atomic spatial coordinates respectively. Next, 3 mutually orthogonal principal axes of the molecule are determined by performing principal component analysis (PCA) on M without dimensionality reduction. This yields 3 principal components (u, v, w) that represent 3 mutually perpendicular molecular axes in the order of decreasing chemical variance from u through w. The original molecular coordinates can now be transformed into the new (u, v, w) coordinate system:

$$R = [ \begin{matrix}{x}_{1}^{′} & {y}_{1}^{′} & {z}_{1}^{′} \\ ⋮ & ⋮ & ⋮ \\ {x}_{n}^{′} & {y}_{n}^{′} & {z}_{n}^{′}\end{matrix} ] = [ \begin{matrix}{x}_{1} & {y}_{1} & {z}_{1} \\ ⋮ & ⋮ & ⋮ \\ {x}_{n} & {y}_{n} & {z}_{n}\end{matrix} ] [ \begin{matrix}{u}_{1} & {v}_{1} & {w}_{1} \\ {u}_{2} & {v}_{2} & {w}_{2} \\ {u}_{3} & {v}_{3} & {w}_{3}\end{matrix} ]$$

A molecular graph with n nodes, each representing a constituent atom can now be constructed by encoding the new transformed atomic coordinates (x′ᵢ, y′ᵢ, z′ᵢ) as node features of the graph. The node features are also appended/prepended by an atomic identifier that encodes the kind of atom that the node represents. This can be either just the atomic number or a one-hot encoding vector of atom type. The molecular graphs so constructed can now be fed as inputs into a GNN. Like the distance matrix approaches, this approach retains rotational, translational and permutational invariance yet its node features scales linearly with system size. Further, the distance matrix does not fully encode 3D molecular shape/topology information while the current approach offers a complete representation of the molecular structure and is therefore expected to be more powerful especially for prediction of intensive molecular properties that tend to require more global descriptors.

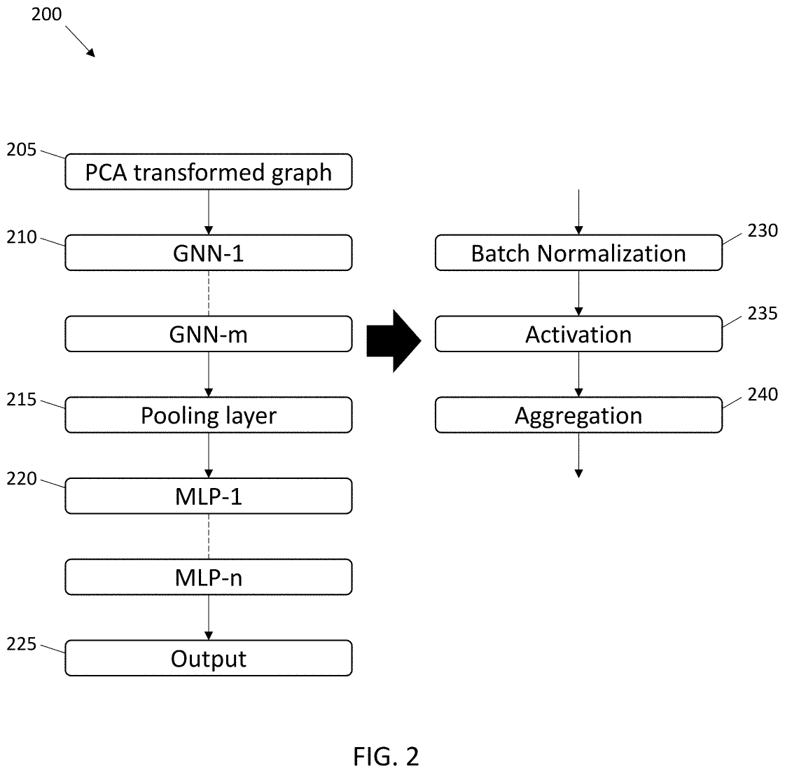

The GNN architecture used in this work is based on the general architecture proposed by You et al.⁸³ and is shown in FIG. 2. The model 200 includes a PCA transformed graph layer 205, a series of GNN layers 210 followed by a pooling layer 215 and a series of classical MLP 220 (Multi-layer Perceptron) layers, and an output layer 225. The model 200 can further include batch normalization 230, activation 235, and aggregation layers 240. The number of GNN 210 (ex. m=6, 8, 10, 12) and MLP layers 220 (ex. n=6, 8, 10, 12) is a hyperparameter in the model, as are the number of nodes (ex. N=512, 1024) in each layer, aggregation functions (ex. sum, average), batch size (ex. 16, 32, 64) and learning rate (ex. 10⁻¹-10⁻³.

In one example, a grid search across hyperparameter size was performed to find the optimal model. The ReLU activation function was used for all neurons in the model. The hyperparameter that yielded the best model for the dataset was as follows: m=12; n=6; N=1024; Batch size=16; Aggregation=Sum; Learning rate=10⁻². The GNN models were built using the Spektral 1.0 library.⁸⁴

### Benchmarking and Validating QM Methods

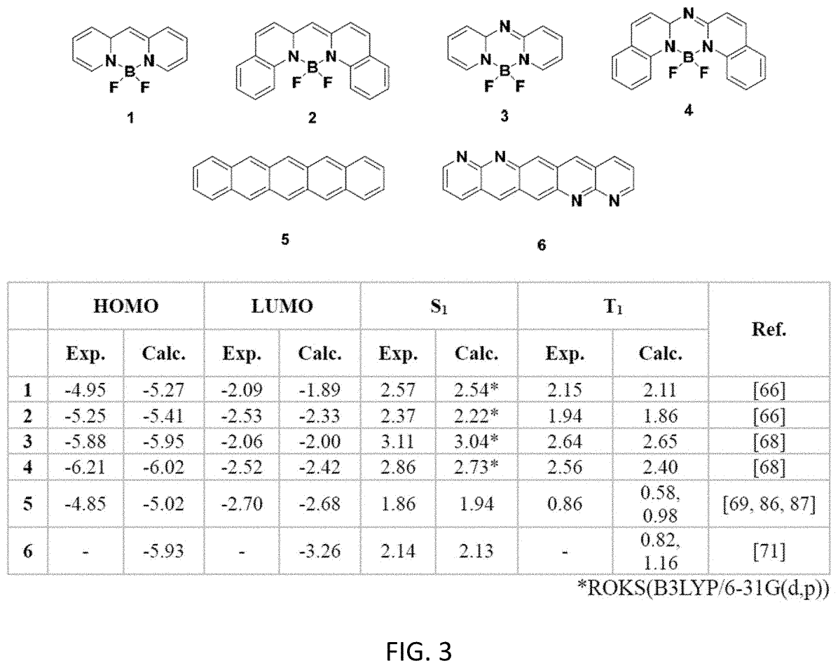

The success of any computational screening/exploration method hinges on how reliably and accurately relevant properties can be computed. With respect to optoelectronic applications, excited state energies (Sₙ, Tₙ) are the most crucial parameters and predicting them with a high level of accuracy is vital. As noted earlier, TDDFT is often the method of choice for computing excited state energies due to its ease of implementation, a balance of low computational cost and high accuracy in most cases. Unfortunately, TDDFT based methods fail to accurately predicted S₁ energies of the cyanine family of dyes to which both the DIPYR and α-DIPYR based compounds belong, with errors in excess of 0.4 eV irrespective of the choice of the functional.^{57,66} For instance, the S₁ energy of BODIPY, a green luminescent dye predicted by TDDFT at the B3LYP/6-31G(d,p) level is 3.1 eV (violet). The errors likely stem from the breakdown of the adiabatic approximation used in traditional TDDFT methods and warrants the need for more sophisticated treatments. ΔSCF methods like MOM (Maximum overlap method) and Restricted Open-shell Kohn Sham method (ROKS) have recently been shown to accurately predict excitation energies in such cases.⁸⁵ Such methods are very attractive since their costs are on par with TDDFT. In one embodiment, the Restricted Open-shell Kohn Sham method (ROKS), which is a ΔSCF method like MOM but is expected to be more reliable in converging to the lowest excited singlet state (S₁), ⁷⁶ was used. ROKS calculations were performed at the B3LYP/6-31G(d,p) level for a series of DIPYR and α-DIPYR based dyes for which experimental data is available and are compared in the table of FIG. 3. The S₁ energies predicted by ROKS are found to be in excellent agreement with the experimental values while TDDFT based methods grossly overestimate the energies. The T₁ energy is another key parameter that should be considered while designing materials for optoelectronic application especially ones like OPVs and OLEDs due to the relevance of triplet-based processes like TTA, TPA, V_{oc} losses in OPVs via triplet channels, luminescence losses in OLEDs through ISC, etc. Additionally, recent reports have indicated that the T₂ state in the DIPYR parent structure is slightly lower in energy than the S₁ state and has been blamed for PLQY losses via ISC into the T₂ state which is further enhanced since the T₂ state bears a different symmetry (El Sayed's rule) relative to the S₁ state.⁶⁶ Therefore, the T₂ state is another parameter that needs to be considered while exploring these compounds for optoelectronic applications. The TDDFT predicted T₁ energies, unlike the S₁ energies are in good agreement with experimental values. Unfortunately, there are no reports that have reported the experimentally measured T₂ energies for any of these systems and hence the TDDFT computed values were relied on. With respect to the pentacene based structures for which experimental data is available, the S₁ energies calculated by TDDFT are found to be in good agreement with experimental values (FIG. 3). However, TDDFT without the Tamm Dancoff approximation (TDA)⁷⁴ is known to underestimate the triplet state energies of pentacene.⁸⁶ Therefore, TDA was used to predict triplet energies for all pentacene-based structures described herein and it can be seen in the table of FIG. 3 that the TDA values are in good agreement with experimental values.

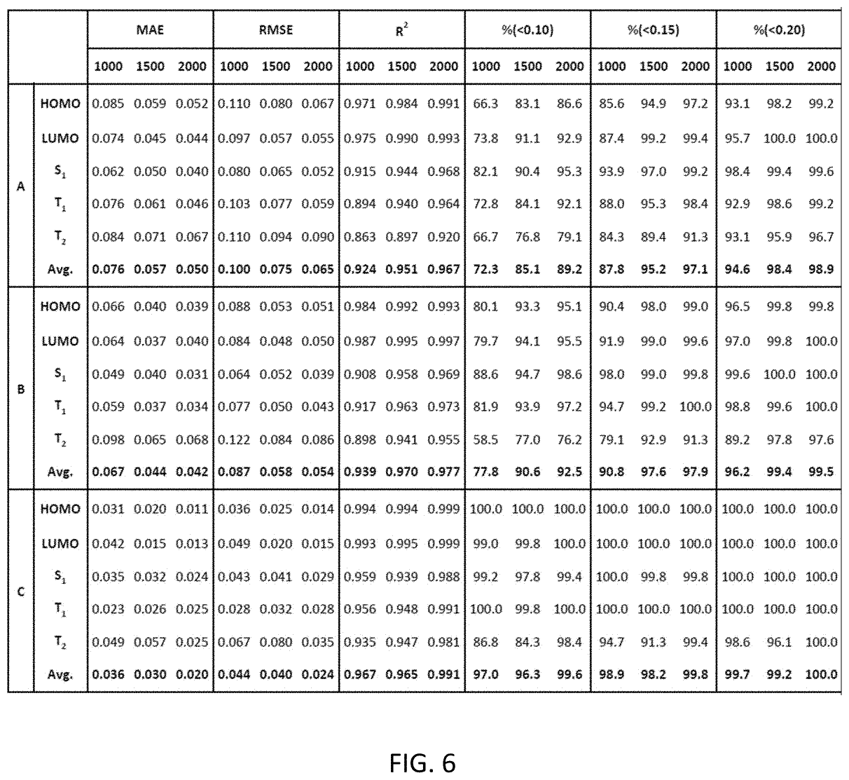

In addition, frontier molecular orbital (HOMO/LUMO) energies are another set of parameters that need to be considered while designing optoelectronics materials. Several studies have benchmarked HOMO/LUMO energies calculated by DFT methods in vacuo against UPS and IPES measurements for a range of organic semiconductor materials and have arrived at linear correlations.^{87,88} For consistency, linear correlations were also derived between the reported UPS/IPES derived HOMO/LUMO values with the DFT computed values at the B3LYP/6-31G(d,p) level, the methodology used in one embodiment and obtain good R² values. While UPS/IPES data is unavailable for DIPYR, α-DIPYR and pentacene-based compounds, electrochemical oxidation and reduction potentials have been reported for a few of these and related compounds and may be used as surrogates. Linear correlations between oxidation/reduction potentials and UPS/IPES derived HOMO/LUMO values have been reported for common organic semiconductors.⁸⁷⁻⁸⁹ The correlation factors reported in Janus et al.⁸⁷ were used in one embodiment. The DFT computed HOMO/LUMO values with the correlation factors (UPS/IPES→DFT) applied are compared with the values derived from electrochemical measurements with the corresponding correlation factors (UPS/IPES→Ox./Red. Potentials) and are found to be in good agreement with each other (FIG. 6). Higher excited states (S₂-S₅ and T3-T₅) though less critical for most optoelectronics applications have also been computed and correspondingly ML models have been developed.

### ML Workflow

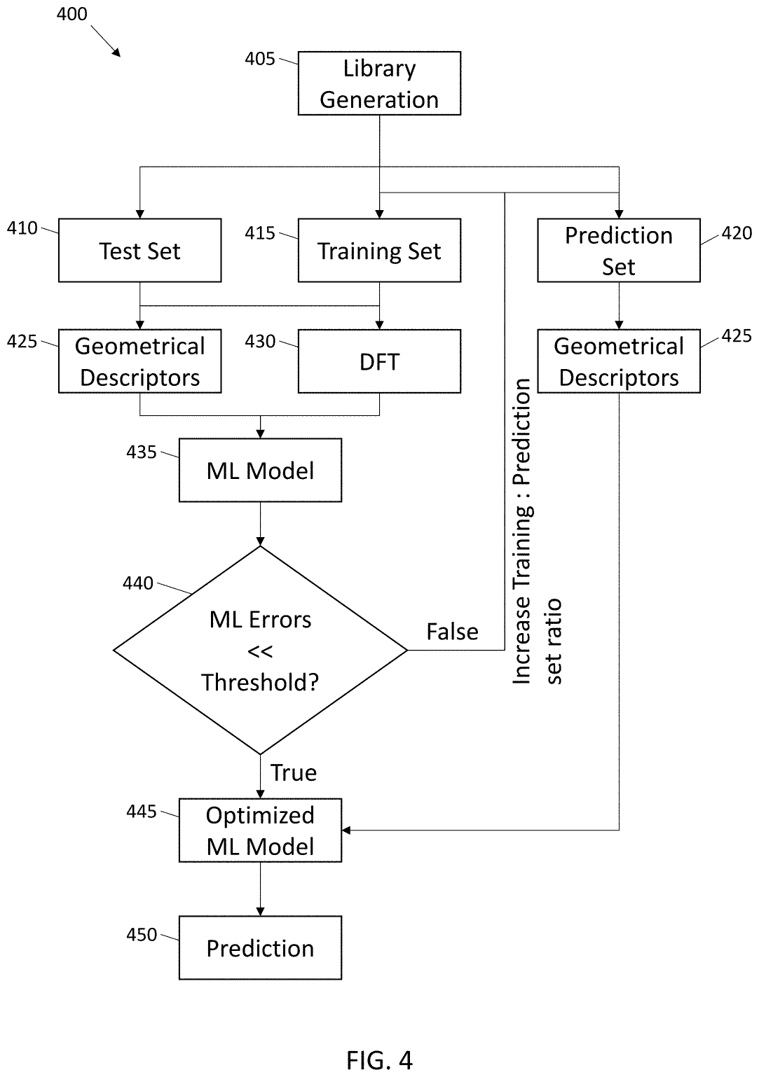

A schematic of the workflow 400 used in this work is shown FIG. 4. Once the libraries are generated 405, the 3D geometry of each molecule is converted to its 12^{NP}3^{B} encoding for the classical KRR and GPR ML models while the GNN models accept 3D cartesian coordinate information as their input (described in the methods section). Each library is split into three sets: a training set 415 which will be used to train the ML models, a test set 410 that will be used to test the validity of the ML models and assess their accuracy, and a prediction set 420 is the rest of the library for which predictions are made using the ML models. DFT calculations 430 parametrized by the aforementioned benchmarks are performed for the molecules in the training and test sets (410, 415) in order to develop the ML models. During training, the ML model 435 takes the geometric descriptor encodings 425 and the DFT computed properties 430 of the molecules in the training set 415 as inputs and attempts to learn the relationship between them. In one embodiment, the size of the training set 415 was initially set to 100 and was gradually increased stepwise in increments of 250 molecules by borrowing molecules from the prediction set 420. At each step, the error metrics 440 (MAE, R²) of the trained ML model are computed for predictions on the test set 410. The training set 415 size may be increased until the desired accuracy is reached or until the error metrics cease to improve appreciably. In one embodiment, the size of the test set 410 was set at 450 molecules for all 3 libraries as further increases in size did not lead to significant differences in the error metrics indicating that a sufficiently representative sampling of the whole library was reached. Finally, the optimized ML model 445 may then be used to make predictions 450 on the rest of the library (prediction set 420).

### Results and Discussion

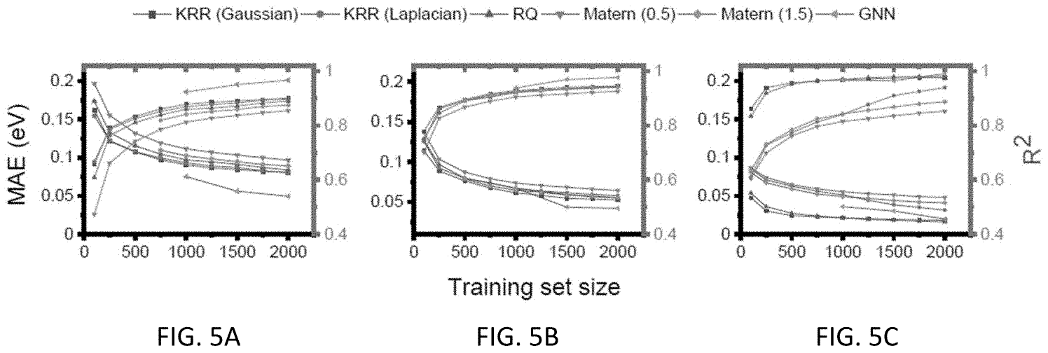

The performance of 2 kernel ridge regression (KRR:Gaussian and KRR:Laplacian) models and 3 gaussian process regression (Rational quadratic and 2 variants of Matern) ML methodologies was compared with the GNN models developed in this work for each of the 3 libraries. Matern (v=1.5) models for libraries A and B failed to converge for training set sizes below 750 and 500 respectively while GNN models were only trained for 1000, 1500 and 2000 training set sizes. The GNN models outperformed the classical ML models and featured the best prediction metrics (MAE and R²) while the Matern (v=0.5) model exhibits the worst metrics among all the models as seen in FIGS. 5A-5C. It should be noted that the metrics reported in FIGS. 5A-5C are averaged across the 5 most pertinent energetic parameters (HOMO, LUMO, S₁, T₁ and T₂).

The GNN models were used for all further analyses as they were the best performing models across the board. The errors follow a normal distribution and expectedly becomes narrower with increasing training set size. In all cases, the error distribution is broadest for the energy of the T₂ state. Library A shows the broadest distribution with about 89.2% and 98.9% of the errors within the 0.1 eV and 0.2 eV bins respectively for the model trained on 2000 samples. This can be attributed the fact that this is the most chemically diverse of all 3 libraries considered. For library B, the model trained on 2000 molecules was able to restrict 92.5% and 99.5% of errors within the 0.1 and 0.2 eV bins respectively on average. For library C on the other hand, the model trained on just 1000 samples was able to confine about 97% of the errors within 0.1 eV. A detailed list of the metrics for individual properties obtained from the models are tabulated in the table of FIG. 6 for the 3 libraries.

For each library, the GNN models trained on 2000 samples were used to make predictions on the rest of the library (prediction set 420). The predictions on A and B indicate that luminophores across the entire visible spectrum may be accessible with S₁ energies spanning 1.3-3.5 eV. Predictions on library C also span a wide parametric design space vis-à-vis OPV applications. The validity of these predictions is demonstrated for two exemplary niche applications discussed below.

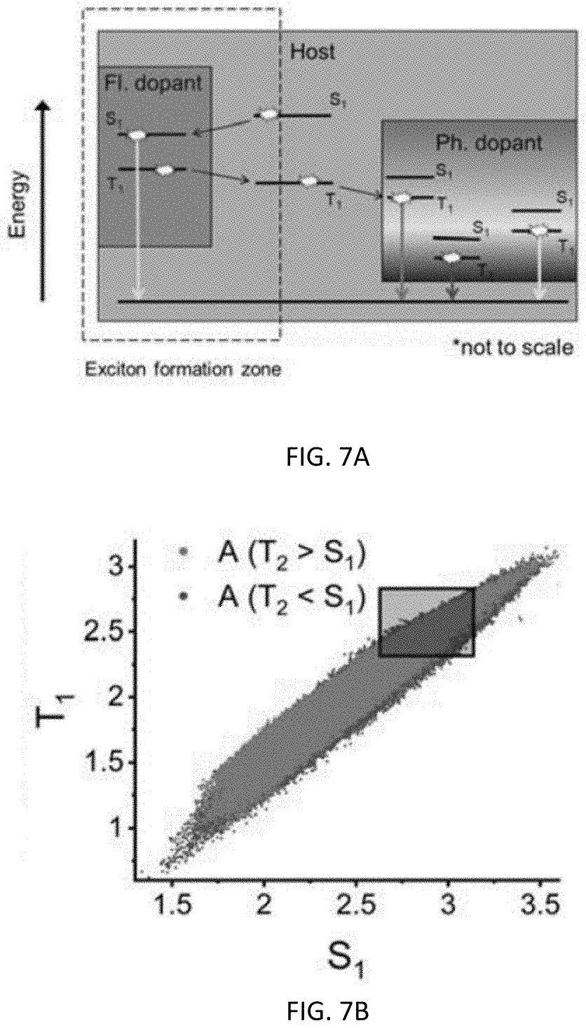

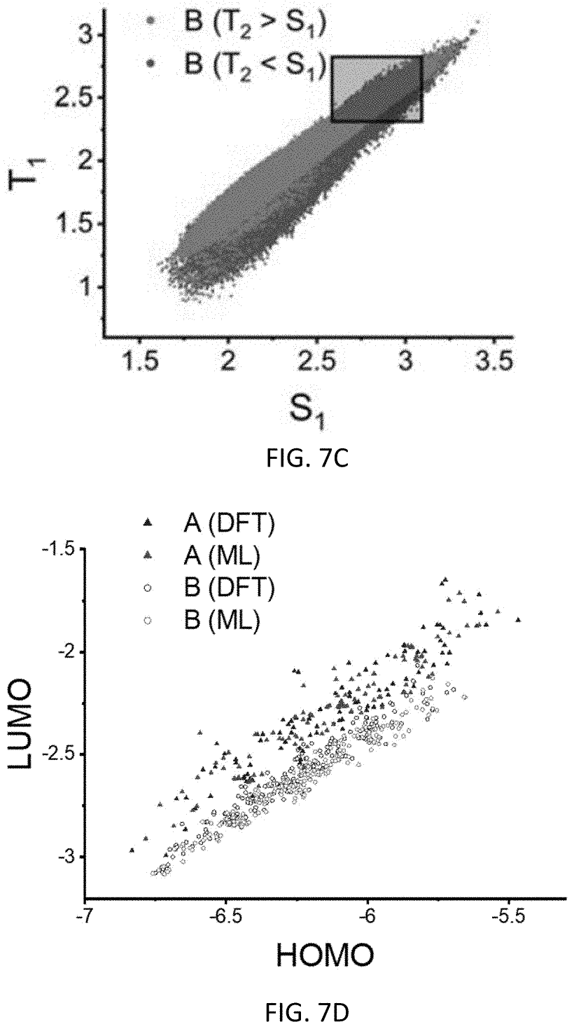

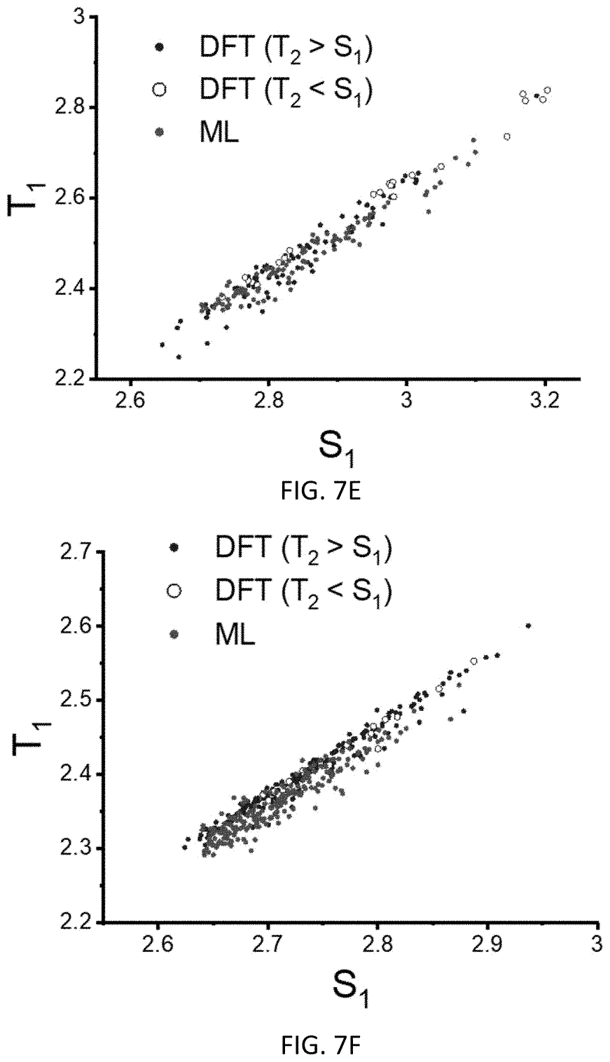

The first exemplary application involves developing an efficient blue fluorophore that has the optimal energetic alignment of energy levels to be viable in a hybrid white-OLED (WOLED) architecture like the one proposed by Sun et al.⁹⁰ The generation of white light for solid-state lighting applications requires red, green and blue-emitting components (or alternatively blue and yellow). The ideal luminophore for these components would be phosphors as they are capable of harvesting both singlet and triplet excitons that are electrogenerated in a 1:3 ratio within the OLED and can therefore reach internal quantum efficiencies as high as 100%.⁹¹⁻⁹⁴ Fluorophores on the other hand can only harvest singlet excitons which caps the maximum IQE achievable at 25%. While several efficient red, green and yellow phosphors have been developed that are stable and have operational lifetimes>10,000 hours, a stable and efficient blue phosphor with a long operational lifetime viable for commercial applications is still elusive. Blue fluorophores can reach longer operational lifetimes necessary for commercial viability but as mentioned earlier are capped at 25% IQE. A hybrid architecture like the one depicted in FIG. 7A which uses a blue fluorophore doped near the exciton formation zone along with red and green (or yellow) phosphors doped a certain distance (greater than the singlet exciton diffusion distance but within that of the triplet excitons) away from the zone within a single stack would in principle be able to achieve white light emission with 100% IQE while eliminating the need for a stable blue phosphor.^{90, 95} This is possible because within such an architecture, provided the energy levels of the components are aligned as depicted, all singlet excitons (25%) formed within the device would be harvested by the blue fluorophore while all the triple excitons (75%) formed would diffuse to the red and green (or yellow) phosphors resulting in emission. The energy level requirements are as follows: The fluorophore should be blue emissive, therefore its S₁ state would preferably be in the 2.64-3.1 eV range while its T₁ state would need to be higher in energy than that of the host which would in turn be higher than that of the green/yellow phosphors used in the device. This translates to the constraint that ideally the T₁ state of the fluorophore be >2.3 eV to ensure that the triplet excitons can diffuse to the phosphors and are not trapped on the fluorophore. Libraries A and B were built with this application in mind as these classes of molecules are known to exhibit very high fluorescence quantum yields with sharp emission lines making them very attractive as dopants. There has been some suggestion from previous reports that the quantum yield can be diminished if the T₂ state lies below the S₁ state in these classes of compounds.⁶⁶ Therefore, the condition that T₂>>S₁ would be an additional criterion that would need to be satisfied by a viable candidate from these libraries. The predictions from the GNN(2000) models for libraries A and B were used to screen for blue fluorophores viable for the current application based on the 3 conditions mentioned above, namely, 2.64<S₁<3.1 eV, T₁>2.3 eV and T₂>S₁. This yields a total of 62,359 and 218,384 candidates from A and B respectively that satisfy these criteria. These were further filtered to include only compounds with 3 or fewer substitutions as these are more attractive from a synthetic standpoint, yielding 751 and 220 candidates, respectively. Of the 751 compounds selected from library A, the top 100 candidates ranked according to their T₂-S₁ gap were chosen for further analysis (i.e. validation by DFT) to keep size of the library manageable while all 220 compounds from B were carried forward to the next step.

DFT calculations as detailed above were then performed on the selected candidates from the previous step to confirm the validity of the ML models and the results are shown in FIGS. 7D, 7E, and 7F. Based on the DFT calculations, 71.0% and 87.7% of the compounds from A and B respectively, predicted by the GNN(2000) ML models were confirmed to satisfy the aforementioned design criteria. It should be noted that analysis of false negatives within the margins were ignored to limit computational overhead and are expected to mirror the false positivity rate due to the symmetric nature of the normal error distribution.

The second exemplary application explored is associated with singlet fission (SF), a phenomenon where upon absorption of a photon, the resulting singlet exciton splits into two triplet excitons.⁶⁹ This usually occurs in molecules whose T₁ state energy is roughly half that of the S₁ state. Singlet fission is very attractive for photovoltaic applications as this enables the utilization of some of the excess energy of high energy excitons (above the junction gap) which would otherwise be lost as heat in a traditional single-junction cell.⁹⁶ SF materials may be used as sensitizers in OPVs or inorganic solar cells to boost efficiency potentially beyond the Shockley-Queisser limit.^{69, 96, 97} Pentacene-based structures are among the few classes of molecules that have been shown to exhibit singlet fission.⁶⁹ From a design standpoint, having a slate of SF materials with a wide range of S₁/T₁ and HOMO/LUMO parameters would enable their incorporation in a range of device configurations and allow for greater flexibility in optimizing for maximal performance. Given the limited number of SF materials that have been identified so far and the desire for a wide gamut of parametric space, Library C, based off the pentacene core was developed.

A viable SF candidate would satisfy the condition that S₁≈2T₁ and more preferably 0<S₁−2T₁<0.2 eV to minimize energy losses. An additional condition: T₂>2T₁ may be imposed to ensure that bimolecular T₁-T₁ annihilation events leading to T₂ excitons are disfavored.^{69,96}

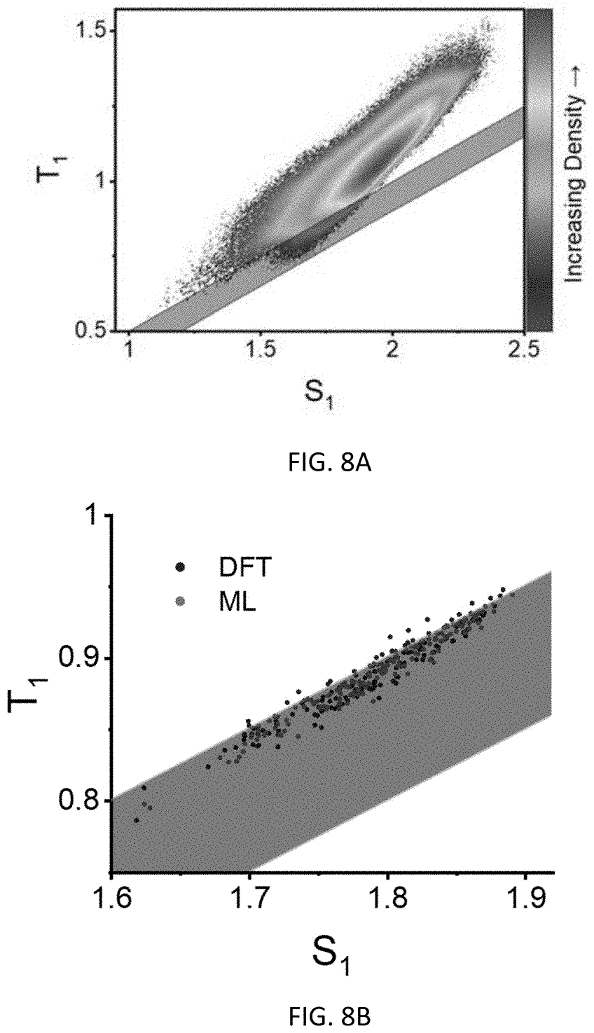

Application of the above constraints to predictions of the GNN(2000) model on library C yields 11,691 SF-likely structures occupying a wide parametric space with HOMO/LUMO energies spanning across a ^{˜}2 eV range with S₁ energies ranging 1.2-2 eV (FIG. 8A). The scope was further narrowed to include only structures with 6 or fewer substitutions yielding a set of 1,935 structures. DFT calculations were performed on a random collection of 150 structures from this set to confirm the validity of the model and the results are depicted in FIG. 8B. Of the 150 structures, 112 (75%) were confirmed by the DFT calculations to satisfy the SF criteria as defined strictly while the rest remain close to the margins as shown in FIG. 8B, demonstrating the efficacy of the model in identifying viable SF candidates.

### Computing Environment

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

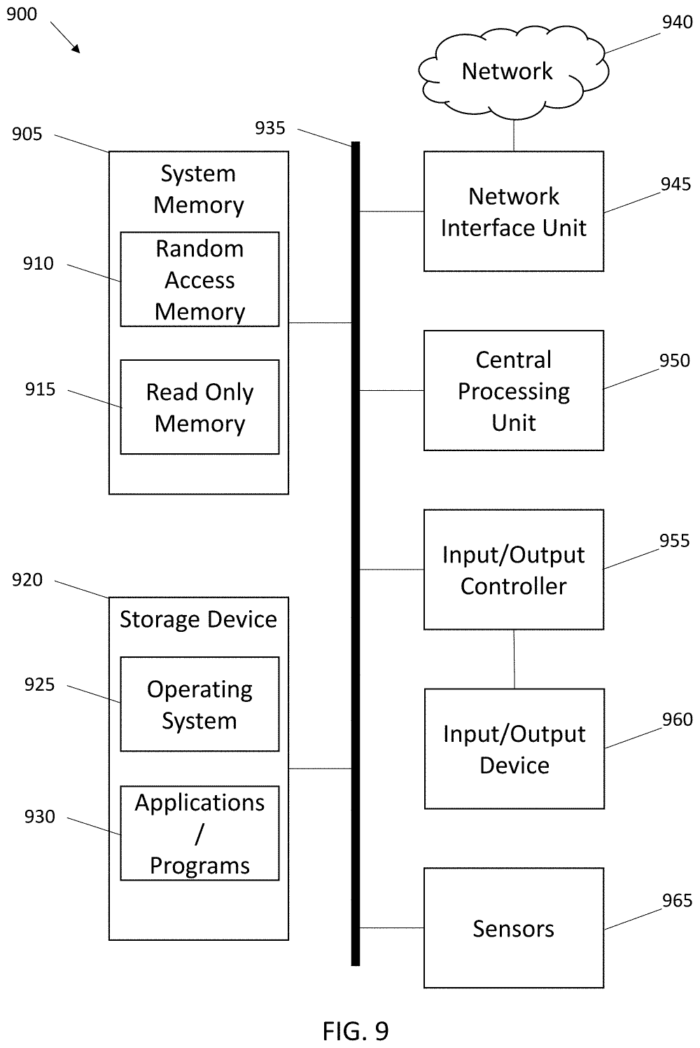

FIG. 9 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 9 depicts an illustrative computer architecture for a computer 900 for practicing the various embodiments of the invention. The computer architecture shown in FIG. 9 illustrates a conventional personal computer, including a central processing unit 950 (“CPU”), a system memory 905, including a random-access memory 910 (“RAM”) and a read-only memory (“ROM”) 915, and a system bus 935 that couples the system memory 905 to the CPU 950. A basic input/output system containing the basic routines that help to transfer information between elements within the computer, such as during startup, is stored in the ROM 915. The computer 900 further includes a storage device 920 for storing an operating system 925, application/program 930, and data.

The storage device 920 is connected to the CPU 950 through a storage controller (not shown) connected to the bus 935. The storage device 920 and its associated computer-readable media, provide non-volatile storage for the computer 900. Although the description of computer-readable media contained herein refers to a storage device, such as a hard disk or CD-ROM drive, it should be appreciated by those skilled in the art that computer-readable media can be any available media that can be accessed by the computer 900.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 900 may operate in a networked environment using logical connections to remote computers through a network 940, such as TCP/IP network such as the Internet or an intranet. The computer 900 may connect to the network 940 through a network interface unit 945 connected to the bus 935. It should be appreciated that the network interface unit 945 may also be utilized to connect to other types of networks and remote computer systems.

The computer 900 may also include an input/output controller 955 for receiving and processing input from a number of input/output devices 960, including a keyboard, a mouse, a touchscreen, a camera, a microphone, a controller, a joystick, or other type of input device. Similarly, the input/output controller 955 may provide output to a display screen, a printer, a speaker, or other type of output device. The computer 900 can connect to the input/output device 960 via a wired connection including, but not limited to, fiber optic, ethernet, or copper wire or wireless means including, but not limited to, Bluetooth, Near-Field Communication (NFC), infrared, or other suitable wired or wireless connections.

As mentioned briefly above, a number of program modules and data files may be stored in the storage device 920 and RAM 910 of the computer 900, including an operating system 925 suitable for controlling the operation of a networked computer. The storage device 920 and RAM 910 may also store one or more applications/programs 930. In particular, the storage device 920 and RAM 910 may store an application/program 930 for providing a variety of functionalities to a user. For instance, the application/program 930 may comprise many types of programs such as a word processing application, a spreadsheet application, a desktop publishing application, a database application, a gaming application, internet browsing application, electronic mail application, messaging application, and the like. According to an embodiment of the present invention, the application/program 930 comprises a multiple functionality software application for providing word processing functionality, slide presentation functionality, spreadsheet functionality, database functionality and the like.

The computer 900 in some embodiments can include a variety of sensors 965 for monitoring the environment surrounding and the environment internal to the computer 900. These sensors 965 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

In conclusion, as described herein, large chemical libraries were combinatorially built based on 3 core structures with the goal of identifying suitable candidates for target optoelectronic applications. QM methods that accurately and cost-effectively predict crucial optoelectronic parameters for the classes of molecules contained in the libraries were identified and benchmarked. Accurate ML models for predicting these optoelectronic parameters were trained based on the benchmarked QM calculations on a fraction (<0.3%) of the library. The predictions from the models were then used to screen the libraries and identify suitable candidates for 2 target applications which were again verified by DFT. It was demonstrated that using the prescriptions presented here, predictive ML models can be obtained for local chemical spaces at the level of accuracy needed to screen and identify suitable candidates for target optoelectronic applications with limited computational resources. While the models presented here already achieve high accuracies using small training sets, future work will be aimed at exploring other types of featurization and ML algorithms that will hopefully achieve even higher data-efficiency and accuracy across more diverse chemical spaces.

The following publications are each hereby incorporated herein by reference in their entirety:
1. Gaultois, M. W.; Oliynyk, A. O.; Mar, A.; Sparks, T. D.; Mulholland, G. J.; Meredig, B., Perspective: Web-based machine learning models for real-time screening of thermoelectric materials properties. Apl Materials 2016, 4 (5), 11.
2. Oliynyk, A. O.; Mar, A., Discovery of Intermetallic Compounds from Traditional to Machine-Learning Approaches. Accounts of Chemical Research 2018, 51 (1), 59-68.
3. Meredig, B.; Agrawal, A.; Kirklin, S.; Saal, J. E.; Doak, J. W.; Thompson, A.; Zhang, K.; Choudhary, A.; Wolverton, C., Combinatorial screening for new materials in unconstrained composition space with machine learning. Physical Review B 2014, 89 (9), 7.
4. Meredig, B.; Antono, E.; Church, C.; Hutchinson, M.; Ling, J. L.; Paradiso, S.; Blaiszik, B.; Foster, I.; Gibbons, B.; Hattrick-Simpers, J.; Mehta, A.; Ward, L., Can machine learning identify the next high-temperature superconductor? Examining extrapolation performance for materials discovery. Molecular Systems Design & Engineering 2018, 3 (5), 819825.
5. Tran, K.; Palizhati, A.; Back, S.; Ulissi, Z. W., Dynamic Workflows for Routine Materials Discovery in Surface Science. Journal of Chemical Information and Modeling 2018, 58 (12), 2392-2400.
6. Jalem, R.; Kimura, M.; Nakayama, M.; Kasuga, T., Informatics-Aided Density Functional Theory Study on the Li Ion Transport of Tavorite-Type L1MTO(4)F (M3+−T5+, Ni2+−T6+). Journal of Chemical Information and Modeling 2015, 55 (6), 1158-1168.
7. Allam, O.; Cho, B. W.; Kim, K. C.; Jang, S. S., Application of DFT-based machine learning for developing molecular electrode materials in Li-ion batteries. Rsc Advances 2018, 8 (69), 39414-39420.
8. Eremin, R. A.; Zolotarev, P. N.; Ivanshina, O. Y.; Bobrikov, I. A., Li(Ni,Co,Al)O-2 Cathode Delithiation: A Combination of Topological Analysis, Density Functional Theory, Neutron Diffraction, and Machine Learning Techniques. Journal of Physical Chemistry C 2017, 121 (51), 28293-28305.
9. Kauwe, S. K.; Rhone, T. D.; Sparks, T. D., Data-Driven Studies of Li-Ion-Battery Materials. Crystals 2019, 9 (1), 9.
10. Ahmad, Z.; Xie, T.; Maheshwari, C.; Grossman, J. C.; Viswanathan, V., Machine Learning Enabled Computational Screening of Inorganic Solid Electrolytes for Suppression of Dendrite Formation in Lithium Metal Anodes. Acs Central Science 2018, 4 (8), 996-1006.
11. Butler, K. T.; Davies, D. W.; Cartwright, H.; Isayev, O.; Walsh, A., Machine learning for molecular and materials science. Nature 2018, 559 (7715), 547-555.
12. Gomez-Bombarelli, R.; Aguilera-Iparraguirre, J.; Hirzel, T. D.; Duvenaud, D.; Maclaurin, D.; Blood-Forsythe, M. A.; Chae, H. S.; Einzinger, M.; Ha, D. G.; Wu, T.; Markopoulos, G.; Jeon, S.; Kang, H.; Miyazaki, H.; Numata, M.; Kim, S.; Huang, W. L.; Hong, S. I.; Baldo, M.; Adams, R. P.; Aspuru-Guzik, A., Design of efficient molecular organic light-emitting diodes by a high-throughput virtual screening and experimental approach. Nature Materials 2016, 15 (10), 1120-+.
13. Unke, O. T.; Meuwly, M., PhysNet: A Neural Network for Predicting Energies, Forces, Dipole Moments, and Partial Charges. Journal of Chemical Theory and Computation 2019, 15 (6), 3678-3693.
14. Schutt, K. T.; Sauceda, H. E.; Kindermans, P. J.; Tkatchenko, A.; Muller, K. R., SchNet—A deep learning architecture for molecules and materials. Journal of Chemical Physics 2018, 148 (24), 11.
15. Ahneman, D. T.; Estrada, J. G.; Lin, S. S.; Dreher, S. D.; Doyle, A. G., Predicting reaction performance in C—N cross-coupling using machine learning. Science 2018, 360 (6385), 186-190.
16. Vasudevan, R. K.; Choudhary, K.; Mehta, A.; Smith, R.; Kusne, G.; Tavazza, F.; Vlcek, L.; Ziatdinov, M.; Kalinin, S. V.; Hattrick-Simpers, J., Materials science in the artificial intelligence age: high-throughput library generation, machine learning, and a pathway from correlations to the underpinning physics. Mrs Communications 2019, 9 (3), 821-838.
17. Sun, W. B.; Zheng, Y. J.; Yang, K.; Zhang, Q.; Shah, A. A.; Wu, Z.; Sun, Y. Y.; Feng, L.; Chen, D. Y.; Xiao, Z. Y.; Lu, S. R.; Li, Y.; Sun, K., Machine learning-assisted molecular design and efficiency prediction for high-performance organic photovoltaic materials. Science Advances 2019, 5 (11), 8.
18. Meftahi, N.; Klymenko, M.; Christofferson, A. J.; Bach, U.; Winkler, D. A.; Russo, S. P., Machine learning property prediction for organic photovoltaic devices. Npj Computational Materials 2020, 6 (1), 8.
19. Mater, A. C.; Coote, M. L., Deep Learning in Chemistry. Journal of Chemical Information and Modeling 2019, 59 (6), 2545-2559.
20. Tanaka, I.; Rajan, K.; Wolverton, C., Data-centric science for materials innovation. Mrs Bulletin 2018, 43 (9), 659-663.
21. Nagasawa, S.; Al-Naamani, E.; Saeki, A., Computer-Aided Screening of Conjugated Polymers for Organic Solar Cell: Classification by Random Forest. Journal of Physical Chemistry Letters 2018, 9 (10), 2639-2646.
22. Butler, K. T.; Frost, J. M.; Skelton, J. M.; Svane, K. L.; Walsh, A., Computational materials design of crystalline solids. Chemical Society Reviews 2016, 45 (22), 6138-6146.
23. Scherbela, M.; Hormann, L.; Jeindl, A.; Obersteiner, V.; Hofmann, O. T., Charting the energy landscape of metal/organic interfaces via machine learning. Physical Review Materials 2018, 2 (4), 9.
24. Elton, D. C.; Boukouvalas, Z.; Butrico, M. S.; Fuge, M. D.; Chung, P. W., Applying machine learning techniques to predict the properties of energetic materials. Scientific Reports 2018, 8, 12.
25. Haghighatlari, M.; Hachmann, J., Advances of machine learning in molecular modeling and simulation. Current Opinion in Chemical Engineering 2019, 23, 51-57.
26. Taylor, R. H.; Rose, F.; Toher, C.; Levy, O.; Yang, K.; Nardelli, M. B.; Curtarolo, S., A RESTful API for exchanging materials data in the AFLOWLIB.org consortium. Computational Materials Science 2014, 93, 178-192.
27. Jain, A.; Ong, S. P.; Hautier, G.; Chen, W.; Richards, W. D.; Dacek, S.; Cholia, S.; Gunter, D.; Skinner, D.; Ceder, G.; Persson, K. A., Commentary: The Materials Project: A materials genome approach to accelerating materials innovation. Apl Materials 2013, 1 (1), 11.
28. Kirklin, S.; Saal, J. E.; Meredig, B.; Thompson, A.; Doak, J. W.; Aykol, M.; Ruhl, S.; Wolverton, C., The Open Quantum Materials Database (OQMD): assessing the accuracy of DFT formation energies. Npj Computational Materials 2015, 1, 15.
29. Zakutayev, A.; Wunder, N.; Schwarting, M.; Perkins, J. D.; White, R.; Munch, K.; Tumas, W.; Phillips, C., An open experimental database for exploring inorganic materials. Scientific Data 2018, 5, 12.
30. Zakutayev, A.; Wunder, N.; Schwarting, M.; Perkins, J. D.; White, R.; Munch, K.; Tumas, W.; C., P., High throughput experimental materials database.
31. Inorganic Crystal Structure Database (ICSD). https://icsd.products.fiz-karlsruhe.de.
32. Friederich, P.; Fediai, A.; Kaiser, S.; Konrad, M.; Jung, N.; Wenzel, W., Toward Design of Novel Materials for Organic Electronics. Advanced Materials 2019, 31 (26), 16.
33. Antono, E.; Matsuzawa, N. N.; Ling, J. L.; Saal, J. E.; Arai, H.; Sasago, M.; Fujii, E., Machine-Learning Guided Quantum Chemical and Molecular Dynamics Calculations to Design Novel Hole-Conducting Organic Materials. Journal of Physical Chemistry A 2020, 124 (40), 8330-8340.
34. Jorgensen, P. B.; Mesta, M.; Shil, S.; Lastra, J. M. G.; Jacobsen, K. W.; Thygesen, K. S.; Schmidt, M. N., Machine learning-based screening of complex molecules for polymer solar cells. Journal of Chemical Physics 2018, 148 (24), 13.
35. Saeki, A.; Kranthiraja, K., A high throughput molecular screening for organic electronics via machine learning: present status and perspective. Japanese Journal of Applied Physics 2020, 59, 10.
36. Gomez-Bombarelli, R.; Aguilera-Iparraguirre, J.; Hirzel, T. D.; Duvenaud, D.; Maclaurin, D.; Blood-Forsythe, M. A.; Chae, H. S.; Einzinger, M.; Ha, D.-G.; Wu, T.; Markopoulos, G.; Jeon, S.; Kang, H.; Miyazaki, H.; Numata, M.; Kim, S.; Huang, W.; Hong, S. I.; Baldo, M.; Adams, R. P.; Aspuru-Guzik, A., Design of efficient molecular organic lightemitting diodes by a high-throughput virtual screening and experimental approach. Nat Mater 2016, 15 (10), 1120-1127.
37. Sahu, H.; Rao, W.; Troisi, A.; Ma, H., Toward Predicting Efficiency of Organic Solar Cells via Machine Learning and Improved Descriptors. Advanced Energy Materials 2018, 8 (24).
38. Atahan-Evrenk, S.; Atalay, F. B., Prediction of Intramolecular Reorganization Energy Using Machine Learning. Journal of Physical Chemistry A 2019, 123 (36), 7855-7863.
39. Hachmann, J.; Olivares-Amaya, R.; Atahan-Evrenk, S.; Amador-Bedolla, C.; SánchezCarrera, R. S.; Gold-Parker, A.; Vogt, L.; Brockway, A. M.; Aspuru-Guzik, A., The Harvard Clean Energy Project: Large-Scale Computational Screening and Design of Organic Photovoltaics on the World Community Grid. The Journal of Physical Chemistry Letters 2011, 2 (17), 2241-2251.
40. Nakata, M.; Shimazaki, T., PubChemQC Project: A Large-Scale First-Principles Electronic Structure Database for Data-Driven Chemistry. Journal of Chemical Information and Modeling 2017, 57 (6), 1300-1308.
41. Blum, L. C.; Reymond, J. L., 970 Million Druglike Small Molecules for Virtual Screening in the Chemical Universe Database GDB-13. Journal of the American Chemical Society 2009, 131 (25), 8732-+.
42. Ramakrishnan, R.; Dral, P. O.; Rupp, M.; von Lilienfeld, O. A., Quantum chemistry structures and properties of 134 kilo molecules. Scientific Data 2014, 1, 7.
43. Ruddigkeit, L.; van Deursen, R.; Blum, L. C.; Reymond, J. L., Enumeration of 166 Billion Organic Small Molecules in the Chemical Universe Database GDB-17. Journal of Chemical Information and Modeling 2012, 52 (11), 2864-2875.
44. Rupp, M.; Tkatchenko, A.; Muller, K. R.; von Lilienfeld, O. A., Fast and Accurate Modeling of Molecular Atomization Energies with Machine Learning. Physical Review Letters 2012, 108 (5), 5.
45. Wu, Z. Q.; Ramsundar, B.; Feinberg, E. N.; Gomes, J.; Geniesse, C.; Pappu, A. S.; Leswing, K.; Pande, V., MoleculeNet: a benchmark for molecular machine learning. Chemical Science 2018, 9 (2), 513-530.
46. Faber, F. A.; Christensen, A. S.; Huang, B.; von Lilienfeld, O. A., Alchemical and structural distribution based representation for universal quantum machine learning. Journal of Chemical Physics 2018, 148 (24), 12.
47. Montavon, G.; Rupp, M.; Gobre, V.; Vazquez-Mayagoitia, A.; Hansen, K.; Tkatchenko, A.; Muller, K. R.; von Lilienfeld, O. A., Machine learning of molecular electronic properties in chemical compound space. New Journal of Physics 2013, 15, 16.
48. Ghosh, K.; Stuke, A.; Todorovic, M.; Jorgensen, P. B.; Schmidt, M. N.; Vehtari, A.; Rinke, P., Deep Learning Spectroscopy: Neural Networks for Molecular Excitation Spectra. Advanced Science 2019, 6 (9), 7.
49. Kang, B.; Seok, C.; Lee, J., Prediction of Molecular Electronic Transitions Using Random Forests. Journal of Chemical Information and Modeling 2020, 60 (12), 5984-5994.
50. Ramakrishnan, R.; Hartmann, M.; Tapavicza, E.; von Lilienfeld, O. A., Electronic spectra from TDDFT and machine learning in chemical space. Journal of Chemical Physics 2015, 143 (8), 8.
51. Jacquemin, D.; Perpete, E. A.; Scuseria, G. E.; Ciofini, I.; Adamo, C., TD-DFT performance for the visible absorption spectra of organic dyes: Conventional versus long-range hybrids. Journal of Chemical Theory and Computation 2008, 4 (1), 123-135.
52. Jacquemin, D.; Wathelet, V.; Perpete, E. A.; Adamo, C., Extensive TD-DFT Benchmark: Singlet-Excited States of Organic Molecules. Journal of Chemical Theory and Computation 2009, 5 (9), 2420-2435.
53. Baer, R.; Livshits, E.; Salzner, U., Tuned Range-Separated Hybrids in Density Functional Theory. Annual Review of Physical Chemistry 2010, 61 (1), 85-109.
54. Stein, T.; Kronik, L.; Baer, R., Prediction of charge-transfer excitations in coumarinbased dyes using a range-separated functional tuned from first principles. Journal of Chemical Physics 2009, 131 (24), 5.
55. Korzdorfer, T.; Sears, J. S.; Sutton, C.; Bredas, J. L., Long-range corrected hybrid functionals for pi-conjugated systems: Dependence of the range-separation parameter on conjugation length. Journal of Chemical Physics 2011, 135 (20), 6.
56. Kronik, L.; Stein, T.; Refaely-Abramson, S.; Baer, R., Excitation Gaps of Finite-Sized Systems from Optimally Tuned Range-Separated Hybrid Functionals. Journal of Chemical Theory and Computation 2012, 8 (5), 1515-1531.
57. Momeni, M. R.; Brown, A., Why Do TD-DFT Excitation Energies of BODIPY/AzaBODIPY Families Largely Deviate from Experiment? Answers from Electron Correlated and Multireference Methods. Journal of Chemical Theory and Computation 2015, 11 (6), 2619-2632.
58. Erten-Ela, S.; Yilmaz, M. D.; Id, B.; Dede, Y.; Id, S.; Akkaya, E. U., A panchromatic boradiazaindacene (BODIPY) sensitizer for dye-sensitized solar cells. Organic Letters 2008, 10 (15), 3299-3302.
59. Chen, J. J.; Conron, S. M.; Erwin, P.; Dimitriou, M.; McAlahney, K.; Thompson, M. E., High-Efficiency BODIPY-Based Organic Photovoltaics. Acs Applied Materials & Interfaces 2015, 7 (1), 662-669.
60. Klfout, H.; Stewart, A.; Elkhalifa, M.; He, H. S., BODIPYs for Dye-Sensitized Solar Cells. Acs Applied Materials & Interfaces 2017, 9 (46), 39873-39889.
61. Zhang, D. K.; Martin, V.; Garcia-Moreno, I.; Costela, A.; Perez-Ojeda, M. E.; Xiao, Y., Development of excellent long-wavelength BODIPY laser dyes with a strategy that combines extending pi-conjugation and tuning ICT effect. Physical Chemistry Chemical Physics 2011, 13 (28), 13026-13033.
62. Marfin, Y. S.; Solomonov, A. V.; Timin, A. S.; Rumyantsev, E. V., Recent Advances of Individual BODIPY and BODIPY-Based Functional Materials in Medical Diagnostics and Treatment. Current Medicinal Chemistry 2017, 24 (25), 2745-2772.
63. Umezawa, K.; Citterio, D.; Suzuki, K., New Trends in Near-Infrared Fluorophores for Bioimaging. Analytical Sciences 2014, 30 (3), 327-349.
64. Ni, Y.; Wu, J. S., Far-red and near infrared BODIPY dyes: synthesis and applications for fluorescent pH probes and bio-imaging. Organic & Biomolecular Chemistry 2014, 12 (23), 3774-3791.
65. Kowada, T.; Maeda, H.; Kikuchi, K., BODIPY-based probes for the fluorescence imaging of biomolecules in living cells. Chemical Society Reviews 2015, 44 (14), 4953-4972.
66. Golden, J. H.; Facendola, J. W.; Sylvinson, M. R. D.; Baez, C. Q.; Djurovich, P. I.; Thompson, M. E., Boron Dipyridylmethene (DIPYR) Dyes: Shedding Light on Pyridine-Based Chromophores. Journal of Organic Chemistry 2017, 82 (14), 7215-7222.
67. Kubota, Y.; Tsuzuki, T.; Funabiki, K.; Ebihara, M.; Matsui, M., Synthesis and Fluorescence Properties of a Pyridomethene-BF2 Complex. Organic Letters 2010, 12 (18), 40104013.
68. Tadle, A. C.; El Roz, K. A.; Soh, C. H.; Ravinson, D. S. M.; Djurovich, P. I.; Forrest, S. R.; Thompson, M. E., Tuning the Photophysical and Electrochemical Properties of AzaBoron-Dipyridylmethenes for Fluorescent Blue OLEDs. Advanced Functional Materials, 8.
69. Smith, M. B.; Michl, J., Singlet Fission. Chemical Reviews 2010, 110 (11), 6891-6936.
70. Halls, M. D.; Djurovich, P. J.; Giesen, D. J.; Goldberg, A.; Sommer, J.; McAnally, E.; Thompson, M. E., Virtual screening of electron acceptor materials for organic photovoltaic applications. New Journal of Physics 2013, 15 (10), 105029.
71. Ukwitegetse, N.; Saris, P. J. G.; Sommer, J. R.; Haiges, R. M.; Djurovich, P. I.; Thompson, M. E., Tetra-Aza-Pentacenes by means of a One-Pot Friedlander Synthesis. Chemistry-a European Journal 2019, 25 (6), 1472-1475.
72. Stewart, J. J. P. MOPAC2016, 2016; Stewart Computational Chemistry, Colorado Springs, Colo., USA.
73. Shao, Y. H.; Gan, Z. T.; Epifanovsky, E.; Gilbert, A. T. B.; Wormit, M.; Kussmann, J.; Lange, A. W.; Behn, A.; Deng, J.; Feng, X. T.; Ghosh, D.; Goldey, M.; Horn, P. R.; Jacobson, L. D.; Kaliman, I.; Khaliullin, R. Z.; Kus, T.; Landau, A.; Liu, J.; Proynov, E. I.; Rhee, Y. M.; Richard, R. M.; Rohrdanz, M. A.; Steele, R. P.; Sundstrom, E. J.; Woodcock, H. L.; Zimmerman, P. M.; Zuev, D.; Albrecht, B.; Alguire, E.; Austin, B.; Beran, G. J. O.; Bernard, Y. A.; Berquist, E.; Brandhorst, K.; Bravaya, K. B.; Brown, S. T.; Casanova, D.; Chang, C. M.; Chen, Y. Q.; Chien, S. H.; Closser, K. D.; Crittenden, D. L.; Diedenhofen, M.; DiStasio, R. A.; Do, H.; Dutoi, A. D.; Edgar, R. G.; Fatehi, S.; Fusti-Molnar, L.; Ghysels, A.; Golubeva-Zadorozhnaya, A.; Gomes, J.; Hanson-Heine, M. W. D.; Harbach, P. H. P.; Hauser, A. W.; Hohenstein, E. G.; Holden, Z. C.; Jagau, T. C.; Ji, H. J.; Kaduk, B.; Khistyaev, K.; Kim, J.; King, R. A.; Klunzinger, P.; Kosenkov, D.; Kowalczyk, T.; Krauter, C. M.; Lao, K. U.; Laurent, A. D.; Lawler, K. V.; Levchenko, S. V.; Lin, C. Y.; Liu, F.; Livshits, E.; Lochan, R. C.; Luenser, A.; Manohar, P.; Manzer, S. F.; Mao, S. P.; Mardirossian, N.; Marenich, A. V.; Maurer, S. A.; Mayhall, N. J.; Neuscamman, E.; Oana, C. M.; Olivares-Amaya, R.; O'Neill, D. P.; Parkhill, J. A.; Perrine, T. M.; Peverati, R.; Prociuk, A.; Rehn, D. R.; Rosta, E.; Russ, N. J.; Sharada, S. M.; Sharma, S.; Small, D. W.; Sodt, A.; Stein, T.; Stuck, D.; Su, Y. C.; Thom, A. J. W.; Tsuchimochi, T.; Vanovschi, V.; Vogt, L.; Vydrov, O.; Wang, T.; Watson, M. A.; Wenzel, J.; White, A.; Williams, C. F.; Yang, J.; Yeganeh, S.; Yost, S. R.; You, Z. Q.; Zhang, I. Y.; Zhang, X.; Zhao, Y.; Brooks, B. R.; Chan, G. K. L.; Chipman, D. M.; Cramer, C. J.; Goddard, W. A.; Gordon, M. S.; Hehre, W. J.; Klamt, A.; Schaefer, H. F.; Schmidt, M. W.; Sherrill, C. D.; Truhlar, D. G.; Warshel, A.; Xu, X.; Aspuru-Guzik, A.; Baer, R.; Bell, A. T.; Besley, N. A.; Chai, J. D.; Dreuw, A.; Dunietz, B. D.; Furlani, T. R.; Gwaltney, S. R.; Hsu, C. P.; Jung, Y. S.; Kong, J.; Lambrecht, D. S.; Liang, W. Z.; Ochsenfeld, C.; Rassolov, V. A.; Slipchenko, L. V.; Subotnik, J. E.; Van Voorhis, T.; Herbert, J. M.; Krylov, A. I.; Gill, P. M. W.; Head-Gordon, M., Advances in molecular quantum chemistry contained in the Q-Chem 4 program package. Molecular Physics 2015, 113 (2), 184-215.
74. Hirata, S.; Head-Gordon, M., Time-dependent density functional theory within the Tamm-Dancoff approximation. Chemical Physics Letters 1999, 314 (3-4), 291-299.
75. Filatov, M.; Shaik, S., A spin-restricted ensemble-referenced Kohn-Sham method and its application to diradicaloid situations. Chemical Physics Letters 1999, 304 (5-6), 429-437.
76. Kowalczyk, T.; Tsuchimochi, T.; Chen, P. T.; Top, L.; Van Voorhis, T., Excitation energies and Stokes shifts from a restricted open-shell Kohn-Sham approach. Journal of Chemical Physics 2013, 138 (16), 8.
77. Rogers, D.; Hahn, M., Extended-Connectivity Fingerprints. Journal of Chemical Information and Modeling 2010, 50 (5), 742-754.
78. Hansen, K.; Biegler, F.; Ramakrishnan, R.; Pronobis, W.; von Lilienfeld, O. A.; Muller, K. R.; Tkatchenko, A., Machine Learning Predictions of Molecular Properties: Accurate Many-Body Potentials and Nonlocality in Chemical Space. Journal of Physical Chemistry Letters 2015, 6 (12), 2326-2331.
79. Collins, C. R.; Gordon, G. J.; von Lilienfeld, O. A.; Yaron, D. J., Constant size descriptors for accurate machine learning models of molecular properties. Journal of Chemical Physics 2018, 148 (24), 11.
80. Pedregosa, F.; Varoquaux, G.; Gramfort, A.; Michel, V.; Thirion, B.; Grisel, O.; Blondel, M.; Prettenhofer, P.; Weiss, R.; Dubourg, V.; Vanderplas, J.; Passos, A.; Cournapeau, D.; Brucher, M.; Perrot, M.; Duchesnay, E., Scikit-learn: Machine Learning in Python. Journal of Machine Learning Research 2011, 12, 2825-2830.
81. Schutt, K. T.; Kessel, P.; Gastegger, M.; Nicoli, K. A.; Tkatchenko, A.; Muller, K. R., SchNetPack: A Deep Learning Toolbox For Atomistic Systems. Journal of Chemical Theory and Computation 2019, 15 (1), 448-455.
82. Cho, H.; Choi, I. S., Enhanced Deep-Learning Prediction of Molecular Properties via Augmentation of Bond Topology. Chemmedchem 2019, 14 (17), 1604-1609.
83. You, J.; Ying, R.; Leskovec, J. In Design Space for Graph Neural Networks, 34th Conference on Neural Information Processing Systems (NeurIPS 2020), Vancouver, Canada, Vancouver, Canada, 2020.
84. Grattarola, D.; Alippi, C., Graph Neural Networks in TensorFlow and Keras with Spektral. Ieee Computational Intelligence Magazine 2021, 16 (1), 99-106.
85. Barca, G. M. J.; Gilbert, A. T. B.; Gill, P. M. W., Simple Models for Difficult Electronic Excitations. Journal of Chemical Theory and Computation 2018, 14 (3), 1501-1509.
86. Zhang, C. R.; Sears, J. S.; Yang, B.; Aziz, S. G.; Coropceanu, V.; Bredas, J. L., Theoretical Study of the Local and Charge-Transfer Excitations in Model Complexes of Pentacene-C-60 Using Tuned Range-Separated Hybrid Functionals. Journal of Chemical Theory and Computation 2014, 10 (6), 2379-2388.
87. Sworakowski, J.; Lipinski, J.; Janus, K., On the reliability of determination of energies of HOMO and LUMO levels in organic semiconductors from electrochemical measurements. A simple picture based on the electrostatic model. Organic Electronics 2016, 33, 300-310.
88. Djurovich, P. I.; Mayo, E. I.; Forrest, S. R.; Thompson, M. E., Measurement of the lowest unoccupied molecular orbital energies of molecular organic semiconductors. Organic Electronics 2009, 10 (3), 515-520.
89. D'Andrade, B. W.; Datta, S.; Forrest, S. R.; Djurovich, P.; Polikarpov, E.; Thompson, M. E.,
Relationship between the ionization and oxidation potentials of molecular organic semiconductors. Organic Electronics 2005, 6 (1), 11-20.
90. Sun, Y. R.; Giebink, N. C.; Kanno, H.; Ma, B. W.; Thompson, M. E.; Forrest, S. R.,
Management of singlet and triplet excitons for efficient white organic light-emitting devices. Nature 2006, 440 (7086), 908-912.
91. Baldo, M.; Lamansky, S.; Burrows, P.; Thompson, M.; Forrest, S., Very high-efficiency green organic light-emitting devices based on electrophosphorescence. Applied Physics Letters 1999, 75, 4.
92. Adachi, C.; Baldo, M. A.; Thompson, M. E.; Forrest, S. R., Nearly 100% internal phosphorescence efficiency in an organic light-emitting device. Journal of Applied Physics 2001, 90, 5048-5051.
93. Baldo, M. A.; O'brien, D.; You, Y.; Shoustikov, A.; Sibley, S.; Thompson, M.; Forrest, S., Highly efficient phosphorescent emission from organic electroluminescent devices. Nature 1998, 395 (6698), 151-154.
94. Adachi, C.; Baldo, M. A.; Forrest, S. R.; Lamansky, S.; Thompson, M. E.; Kwong, R. C., High-efficiency red electrophosphorescence devices. Applied Physics Letters 2001, 78 (11), 1622-1624.
95. Sun, N.; Wang, Q.; Zhao, Y. B.; Chen, Y. H.; Yang, D. Z.; Zhao, F. C.; Chen, J. S.; Ma, D. G., High-Performance Hybrid White Organic Light-Emitting Devices without Interlayer between Fluorescent and Phosphorescent Emissive Regions. Advanced Materials 2014, 26 (10), 1617-1621.
96. Xia, J. L.; Sanders, S. N.; Cheng, W.; Low, J. Z.; Liu, J. P.; Campos, L. M.; Sun, T. L., Singlet Fission: Progress and Prospects in Solar Cells. Advanced Materials 2017, 29 (20), 11.
97. Smith, M. B.; Michl, J., Recent Advances in Singlet Fission. In Annual Review of Physical Chemistry, Vol 64, Johnson, M. A.; Martinez, T. J., Eds. Annual Reviews: Palo Alto, 2013; Vol. 64, pp 361-386.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A method for selecting a material having a desired molecular property for optoelectronic applications, comprising:
generating a combinatorial library of molecule structures derived from a core molecular structure based on a palette of chemical functionalities comprising at least one of a synthetic ease of access to all or most compounds in the generated library, an availability or synthesizability of precursors bearing the most possible combinations of the functionalities, and a chemical disparity or diversity of the functionalities within the palette;
splitting the library into a training set configured to train a graph neural network (GNN) machine learning (ML) model, a test set configured to test the validity of and assess accuracy of the GNN model, and a prediction set where predictions are made using the GNN model;
optimizing geometries of the molecular structures in the training set and test set via a semi-empirical, a molecular mechanics, a density functional theory (DFT), or an ab initio method;
computing ground state and excited state properties via a semi-empirical, a molecular mechanics, a density functional theory (DFT), or an ab initio method;
encoding molecular structure information associated with each molecular structure in the library into a matrix
$$M = [ \begin{matrix}{Z}_{1} {x}_{1} & {Z}_{1} {y}_{1} & {Z}_{1} {z}_{1} \\ ⋮ & ⋮ & ⋮ \\ {Z}_{n} {x}_{n} & {Z}_{n} {y}_{n} & {Z}_{n} {z}_{n}\end{matrix} ]$$
representing the chemical structure in an arbitrary cartesian coordinate system where Zᵢ,xᵢ,yᵢ,zᵢ represent the atomic number, x, y and z atomic spatial coordinates respectively;
determining three mutually orthogonal principal axes (u, v, w) of the molecule by performing principal component analysis (PCA) on M;
transforming the (x, y, z) spatial coordinates into the (u, v, w) mutually orthogonal coordinates via
$$R = [ \begin{matrix}{x}_{1}^{′} & {y}_{1}^{′} & {z}_{1}^{′} \\ ⋮ & ⋮ & ⋮ \\ {x}_{n}^{′} & {y}_{n}^{′} & {z}_{n}^{′}\end{matrix} ] = [ \begin{matrix}{x}_{1} & {y}_{1} & {z}_{1} \\ ⋮ & ⋮ & ⋮ \\ {x}_{n} & {y}_{n} & {z}_{n}\end{matrix} ] [ \begin{matrix}{u}_{1} & {v}_{1} & {w}_{1} \\ {u}_{2} & {v}_{2} & {w}_{2} \\ {u}_{3} & {v}_{3} & {w}_{3}\end{matrix} ] ;$$
constructing a molecular graph with n nodes each representing a constituent atom via encoding the (x′ᵢ, y′ᵢ, z′ᵢ) atomic coordinates as node features of the graph wherein the node features include an atomic identifier that encodes the kind of atom that the node represents;
feeding the molecular graph into the GNN model as an input;
providing the prediction set of molecule structures to the trained GNN model; and
selecting a material having a suitable desired molecular property for optoelectronic applications based on the output of the GNN model.

2. The method of claim 1, further comprising optimizing further the geometries of the molecular structures in the training set and test set via a density functional theory (DFT) method utilizing hybrid functional B3LYP with a 6-31G(d,p) basis set.

3. The method of claim 1, further comprising optimizing further the geometries of the molecular structures in the training set and test set via a quantum chemistry method comprising a low-cost density functional theory (DFT), a Møller-Plesset perturbation theory (MP2), or a coupled cluster method.

4. The method of claim 1, further comprising computing excited state energies of the optimized geometries of the molecular structures via an excited state quantum chemistry method comprising a time-dependent DFT (TDDFT), a Tamm-Dancoff approximation (TDA), an excited state coupled cluster approach, or a ΔSCF approach.

5. The method of claim 1, further comprising computing S₁ energies via a restricted open-shell Kohn Sham (ROKS) ΔSCF approach.

6. The method of claim 1, further comprising performing a grid search across a hyperparameter size to find the optimal model, wherein the hyperparameter comprises a number of GNN layers, a number of MLP layers, a number of nodes, an aggregation function, a batch size, and a learning rate.

7. The method of claim 1, further comprising training via a stepwise approach the GNN model by taking the geometric encodings and the DFT computed properties of the molecules in the training set as inputs to learn the relationship between them.

8. The method of claim 7, further comprising computing at each step the error metrics (MAE, R²) of the trained GNN model to perform predictions on the test set until a desired accuracy is reached or until the error metrics cease to improve appreciably.

9. The method of claim 1, wherein the core molecular structure comprises at least one of boron difluoride aza dipyridylmethene (DIPYR), boron difluoride aza diquinolylmethene (α-azaDIPYR), and Pentacene.

10. The method of claim 1, wherein the palette of chemical functionalities further comprises at least one of a highest occupied molecular orbital (HOMO), a lowest unoccupied molecular orbital (LUMO), an S₁ energy, and a T₁ energy.

11. The method of claim 1, wherein structural information associated with each molecule in the library is encoded into a feature vector to serve as an input to the GNN model, and wherein the feature vector includes at least one of an atom connectivity, a bonding pattern, and a 3D geometry.

12. The method of claim 1, where an effective featurization is learned on the fly during training.

13. The method of claim 1, wherein the (u, v, w) mutually orthogonal coordinates represent 3 mutually perpendicular molecular axes in the order of decreasing chemical variance from u through w.

14. The method of claim 1, wherein the atomic identifier includes the atomic number or a one-hot encoding vector of atom type.

15. The method of claim 1, wherein the node features scales linearly with system size.

16. The method of claim 1, wherein the molecular graph retains rotational, translational and permutational invariance.

17. The method of claim 1, wherein the number of GNN layers is from 1 to 20, the number of MLP layers is from 1 to 20, the number of nodes is from 1 to 2000, the aggregation functions include sums and averages, the batch size is from 1 to 100, and the learning rate is from 1 to 10⁻⁴.

18. The method of claim 1, wherein the size of the training set is from 1 to 500 molecules.

19. A method for selecting a material having a desired molecular property, comprising:
generating a combinatorial library of molecule structures derived from a core molecular structure;
splitting the library into a training set configured to train a graph neural network (GNN) machine learning (ML) model, a test set configured to test the validity of and assess accuracy of the GNN model, and a prediction set where predictions are made using the GNN model;
optimizing geometries of the molecular structures in the training set and test set;
computing excited state energies of the optimized geometries of the molecular structures;
encoding molecular structure information associated with each molecular structure in the library into a matrix
$$M = [ \begin{matrix}{Z}_{1} {x}_{1} & {Z}_{1} {y}_{1} & {Z}_{1} {z}_{1} \\ ⋮ & ⋮ & ⋮ \\ {Z}_{n} {x}_{n} & {Z}_{n} {y}_{n} & {Z}_{n} {z}_{n}\end{matrix} ]$$
representing the chemical structure in an arbitrary cartesian coordinate system where Zᵢ, xᵢ, yᵢ, zᵢ represent the atomic number, x, y and z atomic spatial coordinates respectively;
determining three mutually orthogonal principal axes (u,v,w) of the molecule by performing principal component analysis (PCA) on M;
transforming the (x, y, z) spatial coordinates into the (u, v, w) mutually orthogonal coordinates via
$$R = [ \begin{matrix}{x}_{1}^{′} & {y}_{1}^{′} & {z}_{1}^{′} \\ ⋮ & ⋮ & ⋮ \\ {x}_{n}^{′} & {y}_{n}^{′} & {z}_{n}^{′}\end{matrix} ] = [ \begin{matrix}{x}_{1} & {y}_{1} & {z}_{1} \\ ⋮ & ⋮ & ⋮ \\ {x}_{n} & {y}_{n} & {z}_{n}\end{matrix} ] [ \begin{matrix}{u}_{1} & {v}_{1} & {w}_{1} \\ {u}_{2} & {v}_{2} & {w}_{2} \\ {u}_{3} & {v}_{3} & {w}_{3}\end{matrix} ] ;$$
constructing a molecular graph with n nodes each representing a constituent atom via encoding the (x′ᵢ, y′ᵢ, z′ᵢ) atomic coordinates as node features of the graph wherein the node features include an atomic identifier that encodes the kind of atom that the node represents;
feeding the molecular graph into the GNN model as an input;
providing the prediction set of molecule structures to the trained GNN model; and
selecting a material having a suitable desired molecular property based on the output of the GNN model.

20. A system for selecting a material having a desired molecular property for optoelectronic applications, comprising:
at least one database including data for a plurality of core molecular structures; and
a computing system communicatively connected to the at least one database, comprising a processor and a non-transitory computer-readable medium with instructions stored thereon, which when executed by a processor, perform steps comprising:
generating a combinatorial library of molecule structures derived from a core molecular structure based on a palette of chemical functionalities comprising at least one of a synthetic ease of access to all or most compounds in the generated library, an availability or synthesizability of precursors bearing the most possible combinations of the functionalities, and a chemical disparity or diversity of the functionalities within the palette;
splitting the library into a training set configured to train a graph neural network (GNN) machine learning (ML) model, a test set configured to test the validity of and assess accuracy of the GNN model, and a prediction set where predictions are made using the GNN model;
optimizing geometries of the molecular structures in the training set and test set via a semi-empirical, a molecular mechanics, a density functional theory (DFT), or an ab initio method;
computing ground state and excited state properties via a semi-empirical, a molecular mechanics, a density functional theory (DFT), or an ab initio method;
encoding molecular structure information associated with each molecular structure in the library into a matrix
$$M = [ \begin{matrix}{Z}_{1} {x}_{1} & {Z}_{1} {y}_{1} & {Z}_{1} {z}_{1} \\ ⋮ & ⋮ & ⋮ \\ {Z}_{n} {x}_{n} & {Z}_{n} {y}_{n} & {Z}_{n} {z}_{n}\end{matrix} ]$$
representing the chemical structure in an arbitrary cartesian coordinate system where Zᵢ, xᵢ, yᵢ, zᵢ represent the atomic number, x, y and z atomic spatial coordinates respectively;
determining three mutually orthogonal principal axes (u,v,w) of the molecule by performing principal component analysis (PCA) on M;
transforming the (x, y, z) spatial coordinates into the (u, v, w) mutually orthogonal coordinates via
$$R = [ \begin{matrix}{x}_{1}^{′} & {y}_{1}^{′} & {z}_{1}^{′} \\ ⋮ & ⋮ & ⋮ \\ {x}_{n}^{′} & {y}_{n}^{′} & {z}_{n}^{′}\end{matrix} ] = [ \begin{matrix}{x}_{1} & {y}_{1} & {z}_{1} \\ ⋮ & ⋮ & ⋮ \\ {x}_{n} & {y}_{n} & {z}_{n}\end{matrix} ] [ \begin{matrix}{u}_{1} & {v}_{1} & {w}_{1} \\ {u}_{2} & {v}_{2} & {w}_{2} \\ {u}_{3} & {v}_{3} & {w}_{3}\end{matrix} ] ;$$
constructing a molecular graph with n nodes each representing a constituent atom via encoding the (x′ᵢ, y′ᵢ, z′ᵢ) atomic coordinates as node features of the graph wherein the node features include an atomic identifier that encodes the kind of atom that the node represents;
feeding the molecular graph into the GNN model as an input;
providing the prediction set of molecule structures to the trained GNN model; and
selecting a material having a suitable desired molecular property for optoelectronic applications based on the output of the GNN model.
